(19) United States
(12) Patent Application Publication
(10) Pub. No.: US 2022/0000980 A1
(43) Pub. Date: Jan. 6, 2022

(54) Compositions and Methods for use of CXCL12 in Treatment of Bone Disorders

(71) Applicants: NEW YORK UNIVERSITY, New York, NY (US); UNITED STATES GOVERNMENT AS REPRESENTED BY THE DEPARTMENT OF VETERANS AFFAIRS, Washington, DC (US)

(72) Inventors: Alesha CASTILLO, New York, NY (US); Jin Kim MONTCLARE, New York, NY (US); Kamia PUNIA, Staten Island, NY (US)

(21) Appl. No.: 17/280,297

(22) PCT Filed: Sep. 27, 2019
(86) PCT No.: PCT/US19/53463

§ 371 Date: Mar. 26, 2021

Related U.S. Application Data
(60) Provisional application No. 62/737,164, filed on Sep. 27, 2018.

(51) Int. Cl. A61K 38/19; A61K 31/663; A61K 47/55; A61K 47/54; A61P 19/08

(52) U.S. Cl. CPC A61K 38/195; A61K 31/663; A61P 19/08; A61K 47/54; A61K 47/55

(57) ABSTRACT

The present invention provides compositions and methods for treating a disease or disorder associated with bone defects or reduced or abnormal bone formation.

### CROSS-REFERENCE TO RELATED APPLICATIONS

The present application claims priority to U.S. Provisional Application Ser. No. 62/737,164, filed Sep. 27, 2018, which is incorporated by reference herein in its entirety.

### STATEMENT REGARDING FEDERALLY SPONSORED RESEARCH OR DEVELOPMENT

This invention was made with government support under Grant No. 1101 RX0001500 awarded by U.S. Department of Veterans Affairs. The government has certain rights in the invention.

### BACKGROUND OF THE INVENTION

Bone is a dynamic tissue whose mass and shape are regulated by two processes: bone matrix deposition by osteoblasts and matrix resorption by osteoclasts. In young growing bone, the rate of matrix deposition exceeds the rate of matrix resorption, while in aging bone the rate of resorption typically exceeds formation leading to net bone loss, osteoporosis, and increased fracture risk. Bone provides mechanical support to the body and adapts its shape and size to meet mechanical demands; that is, matrix is preferentially added to the outer surface of bone, or periosteal surface, to optimize its strength (Turner et al. 1995; Lynch et al. 2011). Thus, mechanical stimulation in the form of load bearing exercise (e.g., walking, running, weight lifting) has long been a strategy to maintain bone mass and mechanical integrity of the skeleton throughout life (Office of the Surgeon General (US) 2004).

By mechanisms not yet fully understood, the ability of the aged skeleton to respond to increased mechanical stimulation diminishes (Bassey et al. 1998), and this age-related reduction in mechanoresponsiveness renders exercise less effective in building bone mass (Gomez-Cabello et al. 2012). Understanding the molecular mechanisms that underlie bone formation and resorption, particularly those signals that may be unique to exercise-induced osteogenesis, is critical for the development of novel therapeutic strategies for the prevention and treatment of osteoporosis.

Thus, there is a need in the art for improved compositions and methods for promoting bone formation and treating bone disorders. The present invention satisfies this unmet need.

### SUMMARY OF THE INVENTION

In one aspect, the invention provides methods of disease or disorder associated with bone defects or reduced or abnormal bone formation in a subject in need thereof. In one embodiment, the method comprises administering to the subject a composition comprising a modulator of CXCL12. In one embodiment, the modulator of CXCL12 is at least one selected from the group consisting of a chemical compound, a protein, a peptide, a peptidomemetic, an antibody, a ribozyme, a small molecule chemical compound, a nucleic acid, a vector, an antisense nucleic acid molecule.

In one embodiment, the modulator of CXCL12 is a protein. In one embodiment, the protein comprises at least one sequence of SEQ ID NOs:1-4. In one embodiment, the modulator of CXCL12 is a fusion molecule.

In one embodiment, the fusion molecule comprises (a) CXCL12 or a fragment of CXCL12 and (b) a bisphosphonate. In one embodiment, the fusion molecule comprises a sequence selected from the group consisting of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 15, SEQ ID NO: 15, and SEQ ID NO: 16.

In one embodiment, the bisphosphonate comprises a structure of formula (1):

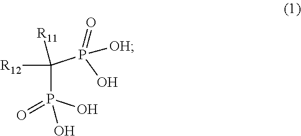

wherein R₁₁ and R₁₂ are each independently selected from the group consisting of hydrogen, halogen, alkyl, aryl, heteroaryl, alkylaryl, alkylheteroaryl, cycloalkyl, heterocyclyl, OR₁₃ and —(CH₂)ₙ—NR₁₃R₁₄;

each occurrence of R₁₃ and R₁₄ is independently selected from the group consisting of hydrogen, alkyl, aryl, heteroaryl, alkylaryl, alkylheteroaryl, cycloalkyl, and heterocycle; and

n is an integer from 0 to 10.

In one embodiment, in formula (1), R₁₁ and R₁₂ are each independently selected from the group consisting of hydrogen, —OH, methyl, Cl, —(CH₂)₂—NH₂, —(CH₂)₃—NH₂, —(CH₂)₅—NH₂, —(CH₂)₂—N(CH₃)((CH₂)₄CH₃),

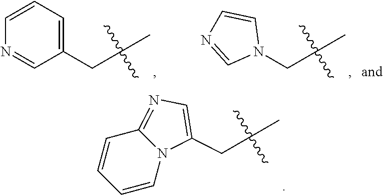 [SMILES: *Cc1cccnc1 ; *Cn1ccnc1 ; *Cc1cnc2ccccn12]

In one embodiment, the bisphosphonate is selected from the group consisting of

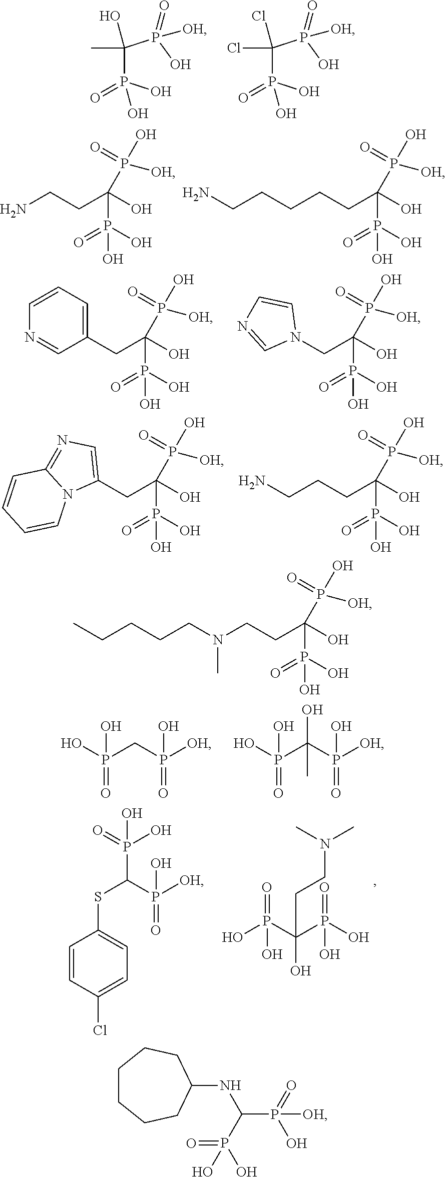

and a salt thereof.

In one embodiment, the disease or disorder is selected from the group consisting of osteoporosis, idiopathic primary osteoporosis, age-related osteoporosis, glucocorticoid-induced osteoporosis, Hajdu-Cheney syndrome, osteolysis, post-transplant bone disease, Paget's disease of bone, bone fracture, periodontal disease, and periodontitis.

In one aspect, the invention provides a fusion molecule. In one embodiment, the fusion molecule comprises (a) CXCL12 or a fragment of CXCL12 and (b) a bisphosphonate. In one embodiment, the fusion molecule comprises a sequence selected from the group consisting of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 15, SEQ ID NO: 15, and SEQ ID NO: 16.

In one embodiment, the bisphosphonate comprises a structure of formula (1):

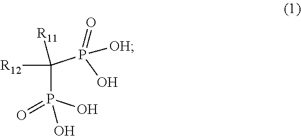

wherein R₁₁ and R₁₂ are each independently selected from the group consisting of hydrogen, halogen, alkyl, aryl, heteroaryl, alkylaryl, alkylheteroaryl, cycloalkyl, heterocyclyl, OR₁₃ and —(CH₂)ₙ—NR₁₃R₁₄;

each occurrence of R₁₃ and R₁₄ is independently selected from the group consisting of hydrogen, alkyl, aryl, heteroaryl, alkylaryl, alkylheteroaryl, cycloalkyl, and heterocycle; and

n is an integer from 0 to 10.

In one embodiment, in formula (1), R₁₁ and R₁₂ are each independently selected from the group consisting of hydrogen, —OH, methyl, Cl, —(CH₂)₂—NH₂, —(CH₂)₃—NH₂, —(CH₂)₅—NH₂, —(CH₂)₂—N(CH₃)((CH₂)₄CH₃),

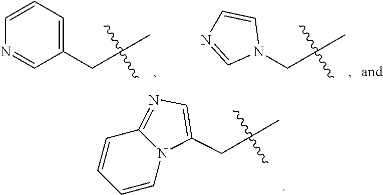 [SMILES: *Cc1cccnc1 ; *Cn1ccnc1 ; *Cc1cnc2ccccn12]

In one embodiment, the bisphosphonate is selected from the group consisting of

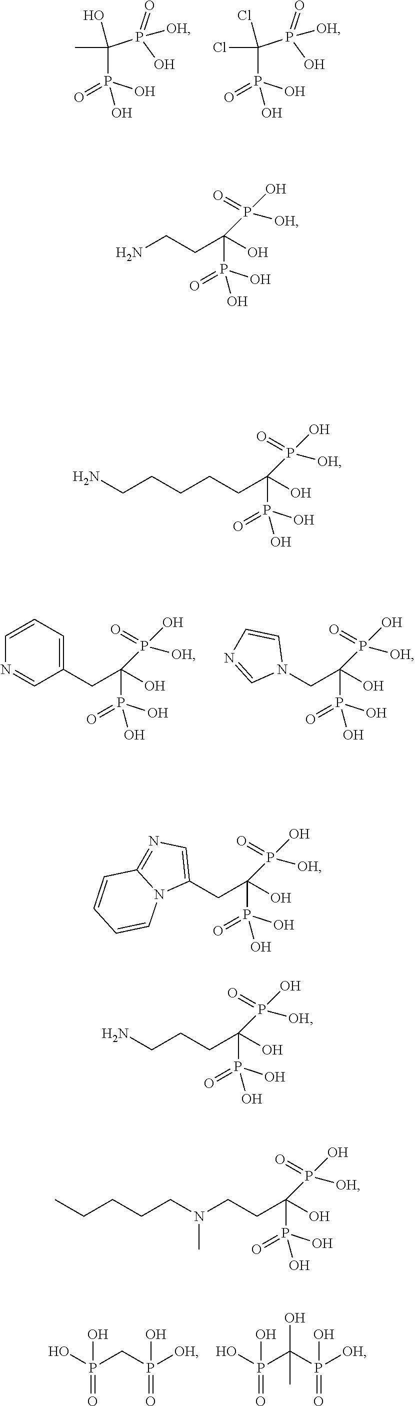

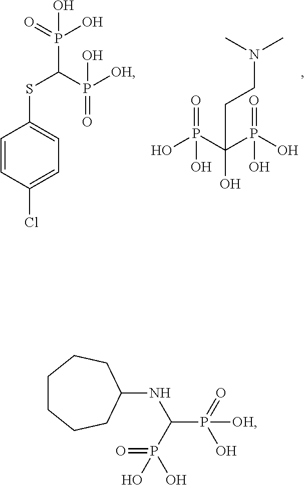 [SMILES: O=P(O)(O)C(Sc1ccc(Cl)cc1)P(=O)(O)O ; CN(C)CCC(O)(P(=O)(O)O)P(=O)(O)O ; O=P(O)(O)C(NC1CCCCCC1)P(=O)(O)O]

and a salt thereof.

In one aspect, the invention provides a method of fusing a CXCL12 or a fragment of CXCL12 to a bisphosphonate. In one embodiment, the method comprises: (a) reacting the N-terminal amine of the CXCL12 or N-terminal amine of the fragment of CXCL12 with 4-ethynylbenzaldehyde to form an alkynyl terminated CXCL12, or incorporating an azidohomoalanine amino acid at the C-terminus of the CXCL12 or the C-terminus of the fragment of CXCL12 to form an alkynyl terminated CXCL12; and (b) conjugating the alkynyl terminated CXCL12 to an azido-bisphosphonate by azide-alkyne cycloaddition, wherein the conjugation of the alkynyl terminated CXCL12 to the azido-bisphosphonate fuses the CXCL12 or the fragment of CXCL12 to the bisphosphonate.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 depicts the results of example experiments demonstrating the quantification of new bone formation from microCT scans of the monocortical defect 10 days after surgery. Bar shows mean and standard deviation N≥3. *P<0.05

FIG. 2, comprising FIG. 2A through FIG. 2F, depicts the results of experiments demonstrating Movat's pentachrome staining of a monocortical tibial defect in mouse. Bone, cartilage, muscle, nuclei, and cytoplasm appear yellow, blue/green, light red, brown, and pink, respectively. The hydrogel appears bright red. The tissues are labeled C: cartilage, B: Bone, M: marrow, and D: defect.

FIG. 3 depicts the results of experiments depicting the confirmation of DMP-1-Cre expression in the growth plate and (panels at left) and in cortical bone. As Cre-recombinase drives deletion of CXCL12 at the level of the single cell, these studies show that CXCL12 deletion is expected in osteocytes present in trabecular and cortical bone in CXCL12fl/fl::DMP-1-Cre mice.

FIG. 4 depicts the results of experiments depicting the confirmation of CXCL12 deletion at the RNA and protein levels.

FIG. 5 depicts the results of experiments demonstrating that no differences were observed in basal periosteal bone formation FIG. 6 depicts the results of experiments demonstrating that no differences were observed in load-strain relationship between CXCL12 knockout and control mice.

FIG. 7 depicts a schematic of an exemplary experimental plan.

FIG. 8, comprising FIG. 8A through FIG. 8E, shows results of load-induced bone formation experiments as depicted by fluorochrome labeling. Representative of (FIG. 8A) non-loaded and (FIG. 8B) loaded periosteal bone. Scale bars=10 μm. Relative (FIG. 8C) MS/BS, (FIG. 8D) MAR and (FIG. 8E) BFR/BS expressed as means±SD.

FIG. 9 depicts the results of example experiments demonstrating the relative periosteal bone formation rates in male and female control and CXCL12 knockout mice.

FIG. 10, comprising FIG. 10A and FIG. 10B, depicts the results of experiments demonstrating that CXCL12 treatment of bones in CXCL12 knockout mice can rescue the bone formation phenotype; that is, bones that were locally treated with CXCL12 exhibited relative periosteal bone formation rates similar to that in non-treated control bones. FIG. 10A depicts experimental results demonstrating CXCL12 deletion in osteocytes results in attenuated load-induced bone formation. FIG. 10B depicts experimental results demonstrating CXCL12 treatment rescues the bone formation phenotype.

FIG. 11 depicts the results of experiments demonstrating that deletion of CXCL12 from primary mouse bone marrow stromal cells were deficient in their ability to undergo osteogenic differentiation.

FIG. 12 depicts experiments demonstrating in vivo transfection of cells located within a bone injury (i.e., externally fixed femoral osteotomy) using AAV-GFP, a pilot study to evaluate an AAV dose-response curve and a precursor to AAV-CXCL12 treatment in vivo.

FIG. 13 depicts the schematic of alkynyl terminated CXCL12 conjugated with azido-PEG-alendronate by azide-alkyne cycloaddition.

### DETAILED DESCRIPTION

The present invention provides compositions and methods for promoting or enhancing bone formation and bone repair. In some aspects, promotion or enhancement of osteogenesis is achieved by modulating CXCL12 activity in osteocytes. For example, it is demonstrated herein that CXCL12 plays a critical role in load-induced bone formation. In certain embodiments, the invention provides a composition comprising a modulator that increases the expression, activity, or both of CXCL12. In some embodiments, the composition comprises CXCL12. The present invention can be used to treat or prevent diseases or disorders associated with bone defects or reduced or abnormal bone formation, including, but not limited to osteoporosis.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “effective amount” of a compound is that amount of compound which is sufficient to provide an effect to the subject or system to which the compound is administered.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Identical” or “identity” as used herein in the context of two or more nucleic acids or polypeptide sequences means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The terms “patient,” “subject,” “individual,” and the like are used interchangeably herein, and refer to any animal, or cells thereof whether in vitro or in situ, amenable to the methods described herein. In some embodiments, the patient, subject or individual is a human.

“Parenteral” administration of a composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A “therapeutic” treatment is a treatment administered to a subject who exhibits signs or symptoms of a disease or disorder, for the purpose of diminishing or eliminating the frequency or severity of those signs or symptoms.

As used herein, “treating a disease or disorder” means reducing the frequency or severity, or both, of at least one sign or symptom of the disease or disorder experienced by a patient.

The phrase “therapeutically effective amount,” as used herein, refers to an amount that is sufficient or effective to prevent or treat (delay or prevent the onset of, prevent the progression of, inhibit, decrease or reverse) a disease or disorder, including alleviating signs and/or symptoms of such diseases and disorders.

To “treat” a disease or disorder as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and including straight, branched chain, or cyclic substituent groups. Examples include methyl, ethyl, propyl, isopropyl, butyl, isobutyl, tert-butyl, pentyl, neopentyl, hexyl, and cyclopropylmethyl.

As used herein, the term “substituted alkyl” means alkyl as defined above, substituted by one, two or three substituents selected from the group consisting of halogen, —OH, alkoxy, —NH₂, amino, azido, —N(CH₃)₂, —C(═O)OH, trifluoromethyl, —C(═O)O(C₁-C₄)alkyl, —C(═O)NH₂, —SO₂NH₂, —C(═NH)NH₂, and —NO₂. Examples of substituted alkyls include, but are not limited to, 2,2-difluoropropyl, 2-carboxycyclopentyl and 3-chloropropyl.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers.

As used herein, the term “halo” or “halogen” alone or as part of another substituent means, unless otherwise stated, a fluorine, chlorine, bromine, or iodine atom.

As used herein, the term “cycloalkyl” refers to a mono cyclic or polycyclic non-aromatic radical, wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to, the following moieties:

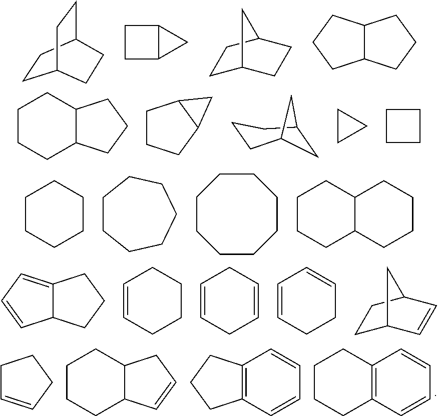

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantane and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon double bond or one carbon triple bond.

As used herein, the term “heterocycloalkyl” or “heterocyclyl” refers to a heteroalicyclic group containing one to four ring heteroatoms each selected from 0, S and N. In one embodiment, each heterocycloalkyl group has from 4 to 10 atoms in its ring system, with the proviso that the ring of said group does not contain two adjacent 0 or S atoms. In another embodiment, the heterocycloalkyl group is fused with an aromatic ring. In one embodiment, the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

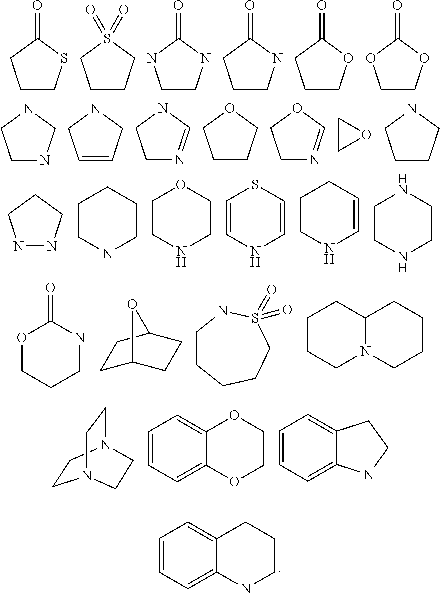

Examples of non-aromatic heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, and naphthyl.

As used herein, the term “aryl-(C₁-C₃)alkyl” means a functional group wherein a one- to three-carbon alkylene chain is attached to an aryl group, e.g., —CH₂CH₂-phenyl. In one embodiment, aryl-(C₁-C₃)alkyl is aryl-CH₂— or aryl-CH(CH₃)—. The term “substituted aryl-(C₁-C₃)alkyl” means an aryl-(C₁-C₃)alkyl functional group in which the aryl group is substituted. Similarly, the term “heteroaryl-(C₁-C₃)alkyl” means a functional group wherein a one to three carbon alkylene chain is attached to a heteroaryl group, e.g., —CH₂CH₂-pyridyl. The term “substituted heteroaryl-(C₁-C₃)alkyl” means a heteroaryl-(C₁-C₃)alkyl functional group in which the heteroaryl group is substituted.

As used herein, the term “heteroaryl” or “heteroaromatic” refers to a heterocycle having aromatic character. A polycyclic heteroaryl may include one or more rings that are partially saturated. Examples include the following moieties:

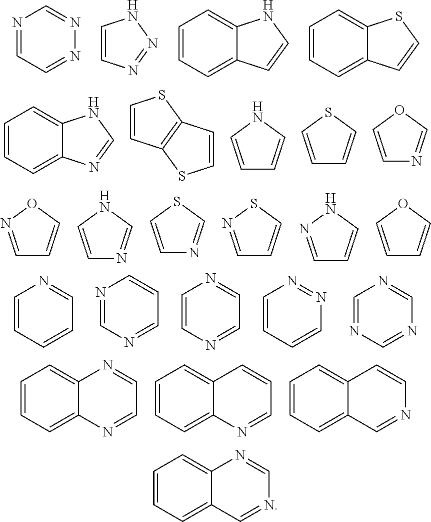

Examples of heteroaryl groups also include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles and heteroaryls include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl.

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In one embodiment, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, —C(═O)NH[substituted or unsubstituted alkyl, or substituted or unsubstituted phenyl], —C(═O)N[H or alkyl]2, —OC(═O)N[substituted or unsubstituted alkyl]2, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]2, and —C(NH₂)[substituted or unsubstituted alkyl]2. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, —ON(O)₂, and —C(═O)OH. In yet one embodiment, the substituents are independently selected from the group consisting of C₁₋₆ alkyl, —OH, C₁₋₆ alkoxy, halo, amino, acetamido, oxo and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

In one embodiment, the invention provides compositions comprising a modulator of CXCL12 expression or activity. In some embodiments, the composition activates, increases or enhances CXCL12 expression or activity. In some embodiments, the modulator comprises any compound, molecule, or agent that activates, increases, or enhances expression or activity. In some embodiments, the modulator comprises nucleic acid molecule, a peptide, a small molecule, a siRNA, a ribozyme, an antisense nucleic acid, an antagonist, an aptamer, a peptidomimetic, or any combination thereof.

In some embodiments, the modulator comprises CXCL12. In some embodiments, the modulator comprises a fusion molecule comprising a targeting domain and a modulator domain, wherein the modulator domain comprises CXCL12, or a fragment or a variant thereof. In some embodiments, the targeting domain of the fusion molecule comprises a bisphosphonate or analog thereof.

In one aspect, the present invention provides a method for modulating CXCL12 expression or activity in a cell, such as an osteocyte or bone-lining cell. In one embodiment, the present invention provides methods for enhancing or promoting osteogenesis. In some embodiments, the methods are used to treat or prevent a disease or disorder associated with bone defects or reduced or abnormal bone formation. Exemplary diseases and disorders include, but are not limited to, osteoporosis, idiopathic primary osteoporosis, age-related osteoporosis, post-menopausal osteoporosis, glucocorticoid-induced osteoporosis, disuse-related bone loss, Hajdu-Cheney syndrome, osteolysis, and post-transplant bone disease, Paget's disease of bone, bone fracture, periodontal disease, and periodontitis.

### Compositions

In one embodiment, the present invention provides compositions for modulating CXCL12. In some embodiments, the composition activates, increases or enhances CXCL12 expression, activity, or both. The present invention is based in part upon the discovery that CXCL12 is a critical regulator of osteogenesis. In certain instances, CXCL12 mediates recruitment of osteogenic cells, proliferation of resident stem cells, differentiation of stem cells into osteoblasts, and/or inhibition of apoptosis.

In various embodiments, the composition comprises a modulator of CXCL12. In some embodiments, the modulator of CXCL12 is any compound, molecule, or agent that activates, increases or enhances the expression, activity, or function of CXCL12. In various embodiments, the modulator of CXCL12 comprises a nucleic acid molecule, a peptide, a small molecule, a siRNA, a ribozyme, an antisense nucleic acid, an antagonist, an aptamer, a peptidomimetic, or any combination thereof.

It is understood by one skilled in the art, that an increase in the level of CXCL12 encompasses the increase of CXCL12 protein expression. Additionally, the skilled artisan would appreciate, that an increase in the level of CXCL12 includes an increase in CXCL12 activity. Thus, increasing the level or activity of CXCL12 includes, but is not limited to, increasing transcription, translation, or both, of a nucleic acid encoding CXCL12; and it also includes increasing any activity of CXCL12 as well.

Activation of CXCL12 can be assessed using a wide variety of methods, including those disclosed herein, as well as methods well-known in the art or to be developed in the future. That is, the routineer would appreciate, based upon the disclosure provided herein, that increasing the level or activity of CXCL12 can be readily assessed using methods that assess the level of a nucleic acid encoding CXCL12 (e.g., mRNA) and/or the level of CXCL12 polypeptide in a biological sample obtained from a subject.

A CXCL12 activator can include, but should not be construed as being limited to, a chemical compound, a protein, a peptidomemetic, an antibody, a nucleic acid molecule. One of skill in the art would readily appreciate, based on the disclosure provided herein, that a CXCL12 activator encompasses a chemical compound that increases the level, enzymatic activity, or the like of CXCL12. Additionally, a CXCL12 activator encompasses a chemically modified compound, and derivatives, as is well known to one of skill in the chemical arts.

It will be understood by one skilled in the art, based upon the disclosure provided herein, that an increase in the level of CXCL12 encompasses the increase in CXCL12 expression, including transcription, translation, or both. The skilled artisan will also appreciate, once armed with the teachings of the present invention, that an increase in the level of CXCL12 includes an increase in CXCL12 activity (e.g., enzymatic activity, receptor binding activity, etc.). Thus, increasing the level or activity of CXCL12 includes, but is not limited to, increasing the amount of CXCL12 polypeptide, increasing transcription, translation, or both, of a nucleic acid encoding CXCL12; and it also includes increasing any activity of a CXCL12 polypeptide as well. The CXCL12 activator compositions and methods of the invention can selectively activate CXCL12. Thus, the present invention relates to treating or preventing a disease or disorder associated with bone defects or reduced or abnormal bone formation by administration of a CXCL12 polypeptide, a recombinant CXCL12 polypeptide, an active CXCL12 polypeptide fragment, or an activator of CXCL12 expression or activity.

Further, one of skill in the art would, when equipped with this disclosure and the methods exemplified herein, appreciate that a CXCL12 activator includes such activators as discovered in the future, as can be identified by well-known criteria in the art of pharmacology, such as the physiological results of activation of CXCL12 as described in detail herein and/or as known in the art. Therefore, the present invention is not limited in any way to any particular CXCL12 activator as exemplified or disclosed herein; rather, the invention encompasses those activators that would be understood by the routineer to be useful as are known in the art and as are discovered in the future.

Further methods of identifying and producing a CXCL12 activator are well known to those of ordinary skill in the art, including, but not limited, obtaining an activator from a naturally occurring source. Alternatively, a CXCL12 activator can be synthesized chemically. Further, the routineer would appreciate, based upon the teachings provided herein, that a CXCL12 activator can be obtained from a recombinant organism. Compositions and methods for chemically synthesizing CXCL12 activators and for obtaining them from natural sources are well known in the art and are described in the art.

One of skill in the art will appreciate that an activator can be administered as a small molecule chemical, a protein, a nucleic acid construct encoding a protein, or combinations thereof. Numerous vectors and other compositions and methods are well known for administering a protein or a nucleic acid construct encoding a protein to cells or tissues. Therefore, the invention includes a method of administering a protein or a nucleic acid encoding a protein that is an activator of CXCL12.

One of skill in the art will realize that diminishing the amount or activity of a molecule that itself diminishes the amount or activity of CXCL12 can serve to increase the amount or activity of CXCL12. Any inhibitor of a regulator of CXCL12 is encompassed in the invention. As a non-limiting example, antisense is described as a form of inhibiting a regulator of CXCL12 in order to increase the amount or activity of CXCL12. Antisense oligonucleotides are DNA or RNA molecules that are complementary to some portion of a mRNA molecule. When present in a cell, antisense oligonucleotides hybridize to an existing mRNA molecule and inhibit translation into a gene product. Inhibiting the expression of a gene using an antisense oligonucleotide is well known in the art (Marcus-Sekura, 1988, Anal. Biochem. 172:289), as are methods of expressing an antisense oligonucleotide in a cell (Inoue, U.S. Pat. No. 5,190,931). The methods of the invention include the use of antisense oligonucleotide to diminish the amount of a molecule that causes a decrease in the amount or activity CXCL12, thereby increasing the amount or activity of CXCL12. Contemplated in the present invention are antisense oligonucleotides that are synthesized and provided to the cell by way of methods well known to those of ordinary skill in the art. As an example, an antisense oligonucleotide can be synthesized to be between about 10 and about 100, or between about 15 and about 50 nucleotides long. The synthesis of nucleic acid molecules is well known in the art, as is the synthesis of modified antisense oligonucleotides to improve biological activity in comparison to unmodified antisense oligonucleotides (Tullis, 1991, U.S. Pat. No. 5,023,243).

Similarly, the expression of a gene may be inhibited by the hybridization of an antisense molecule to a promoter or other regulatory element of a gene, thereby affecting the transcription of the gene. Methods for the identification of a promoter or other regulatory element that interacts with a gene of interest are well known in the art, and include such methods as the yeast two hybrid system (Bartel and Fields, eds., In: The Yeast Two Hybrid System, Oxford University Press, Cary, N.C.).

Alternatively, inhibition of a gene expressing a protein that diminishes the level or activity of CXCL12 can be accomplished through the use of a ribozyme. Using ribozymes for inhibiting gene expression is well known to those of skill in the art (see, e.g., Cech et al., 1992, J. Biol. Chem. 267:17479; Hampel et al., 1989, Biochemistry 28: 4929; Altman et al., U.S. Pat. No. 5,168,053). Ribozymes are catalytic RNA molecules with the ability to cleave other single-stranded RNA molecules. Ribozymes are known to be sequence specific, and can therefore be modified to recognize a specific nucleotide sequence (Cech, 1988, J. Amer. Med. Assn. 260:3030), allowing the selective cleavage of specific mRNA molecules. Given the nucleotide sequence of the molecule, one of ordinary skill in the art could synthesize an antisense oligonucleotide or ribozyme without undue experimentation, provided with the disclosure and references incorporated herein.

One of skill in the art will appreciate that a CXCL12 polypeptide, a recombinant CXCL12 polypeptide, or an active CXCL12 polypeptide fragment can be administered singly or in any combination thereof. Further, a CXCL12 polypeptide, a recombinant CXCL12 polypeptide, or an active CXCL12 polypeptide fragment can be administered singly or in any combination thereof in a temporal sense, in that they may be administered simultaneously, before, and/or after each other. One of ordinary skill in the art will appreciate, based on the disclosure provided herein, that a CXCL12 polypeptide, a recombinant CXCL12 polypeptide, or an active CXCL12 polypeptide fragment can be used to prevent or treat a disease or disorder associated with bone defects or reduced or abnormal bone formation, and that an activator can be used alone or in any combination with another CXCL12 polypeptide, recombinant CXCL12 polypeptide, active CXCL12 polypeptide fragment, or CXCL12 activator to effect a therapeutic result.

One of skill in the art, when armed with the disclosure herein, would appreciate that treating or preventing a disease or disorder associated with bone defects or reduced or abnormal bone formation encompasses administering to a subject a CXCL12 polypeptide, a recombinant CXCL12 polypeptide, an active CXCL12 polypeptide fragment, or CXCL12 activator as a preventative measure against a disease or disorder associated with bone defects or reduced or abnormal bone formation uptake. As more fully discussed elsewhere herein, methods of increasing the level or activity of a CXCL12 encompass a wide plethora of techniques for increasing not only CXCL12 activity, but also for increasing expression of a nucleic acid encoding CXCL12. Additionally, as disclosed elsewhere herein, one skilled in the art would understand, once armed with the teaching provided herein, that the present invention encompasses a method of preventing a wide variety of diseases where increased expression and/or activity of CXCL12 mediates, treats or prevents the disease. Further, the invention encompasses treatment or prevention of such diseases discovered in the future.

The invention encompasses administration of a CXCL12 polypeptide, a recombinant CXCL12 polypeptide, an active CXCL12 polypeptide fragment, or a CXCL12 activator to practice the methods of the invention; the skilled artisan would understand, based on the disclosure provided herein, how to formulate and administer the appropriate CXCL12 polypeptide, recombinant CXCL12 polypeptide, active CXCL12 polypeptide fragment, or CXCL12 activator to a subject. However, the present invention is not limited to any particular method of administration or treatment regimen. This is especially true where it would be appreciated by one skilled in the art, equipped with the disclosure provided herein, including the reduction to practice using an art-recognized models of bone defects or reduced or abnormal bone formation, that methods of administering a CXCL12 polypeptide, a recombinant CXCL12 polypeptide, an active CXCL12 polypeptide fragment, or CXCL12 activator can be determined by one of skill in the pharmacological arts.

As used herein, the term “pharmaceutically-acceptable carrier” means a chemical composition with which an appropriate CXCL12 polypeptide, recombinant CXCL12 polypeptide, active CXCL12 polypeptide fragment, or CXCL12 activator, may be combined and which, following the combination, can be used to administer the appropriate CXCL12 polypeptide, recombinant CXCL12 polypeptide, active CXCL12 polypeptide fragment, or CXCL12 activator to a subject.

In some embodiments, the modulator comprises CXCL12. In some embodiments, the modulator comprises a fusion molecule comprising a targeting domain and a modulator domain, wherein the modulator domain comprises CXCL12, or a fragment or a variant thereof. In some embodiments, the targeting domain of the fusion molecule comprises a bisphosphonate or analog thereof.

### Small Molecule Inhibitors

In some embodiments, the modulator comprises a small molecule. When the modulator comprises a small molecule, a small molecule may be obtained using standard methods known to the skilled artisan. Such methods include chemical organic synthesis or biological means. Biological means include purification from a biological source, recombinant synthesis and in vitro translation systems, using methods well known in the art. In one embodiment, a small molecule comprises an organic molecule, inorganic molecule, biomolecule, synthetic molecule, and the like.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

The small molecule and small molecule compounds described herein may be present as salts even if salts are not depicted and it is understood that the invention embraces all salts and solvates of the inhibitors depicted here, as well as the non-salt and non-solvate form of the inhibitors, as is well understood by the skilled artisan. In some embodiments, the salts of the inhibitors of the invention are pharmaceutically acceptable salts.

Where tautomeric forms may be present for any of the inhibitors described herein, each and every tautomeric form is intended to be included in the present invention, even though only one or some of the tautomeric forms may be explicitly depicted. For example, when a 2-hydroxypyridyl moiety is depicted, the corresponding 2-pyridone tautomer is also intended.

The invention also includes any or all of the stereochemical forms, including any enantiomeric or diasteriomeric forms of the inhibitors described. The recitation of the structure or name herein is intended to embrace all possible stereoisomers of small molecule depicted. All forms of the small molecule are also embraced by the invention, such as crystalline or non-crystalline forms of the small molecule. Compositions comprising a small molecule of the invention are also intended, such as a composition of substantially pure small molecule, including a specific stereochemical form thereof, or a composition comprising mixtures of small molecules of the invention in any ratio, including two or more stereochemical forms, such as in a racemic or non-racemic mixture.

In one embodiment, the small molecule of the invention comprises an analog or derivative of a small molecule described herein.

In one embodiment, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecules described herein are derivatized/analoged as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be homocycles or heterocycles.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule inhibitors described herein or can be based on a scaffold of a small molecule inhibitor described herein, but differing from it in respect to certain components or structural makeup, which may have a similar or opposite action metabolically. An analog or derivative of any of a small molecule in accordance with the present invention can be used to treat a disease or disorder associated with bone defects or reduced or abnormal bone formation.

In one embodiment, the small molecules described herein can independently be derivatized/analoged by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

### Proteins and Peptides

In one embodiment, the composition comprises a peptide that activates, increases, or enhances CXCL12 expression, activity or both.

In one embodiment, the peptide comprises CXCL12, or a fragment or derivative thereof.

In certain embodiments, the CXCL12 of the composition can be any isoform, in its cleaved or uncleaved state, of CXCL12 including, but not limited to: CXCL12α (1-68), CXCL12β (1-72), CXCL12α(1-67), CXCL12α(3-67), CXCL12α(5-67), CXCL12β(1-72), CXCL12β(3-72), CXCL12β(5-72), CXCL12γ, CXCL12δ, CXCL12ε, CXCL12φ, CXCL12 isoform 7, and the like.

In one embodiment, CXCL12 of the composition comprises CXCL12α, having comprising the amino acid sequence of:

(SEQ ID NO: 1)
KPVSLSYRCPCRFFESHVARANVKHLKILNTPNCALQIVARLKNNNRQVC
IDPKLKWIQEYLEKALNK.

In certain embodiments, the CXCL12 of the composition comprises a CXCL12 mutant or variant, wherein one or more serine residues are mutated to cysteine or threonine to prevent CXCL12 cleavage. For example, in one embodiment, the CXCL12 variant comprises the amino acid sequence of:

(SEQ ID NO: 2)
KPVCLCYRCPCRFFESHVARANVKHLKILNTPNCALQIVARLKNNNRQVC
IDPKLKWIQEYLEKALNK.

In one embodiment, the CXCL12 variant comprises the amino acid sequence of:

(SEQ ID NO: 3)
KPLCVCYRCPCRFFESHVARANVKHLKILNTPNCALQIVARLKNNNRQVC
IDPKLKWIQEYLEKALNK.

In one embodiment, the composition comprises a biologically functional fragment of CXCL12, for example the receptor binding domain of CXCL12. As would be understood in the art, a biologically functional fragment is a portion or portions of a full-length sequence that retains a biological function of the full-length sequence.

In some embodiments, the composition comprises CXCL12 having a signal peptide, including but not limited to the signal peptide of:

| | |
|---|---|
| MNAKVVVVLVLVLTALCLSDG. | (SEQ ID NO: 4) |

The invention should also be construed to include any form of a peptide variant having substantial homology to an amino acid sequence disclosed herein. In one embodiment, a protein variant is at least about 50% homologous, at least about 70% homologous, at least about 80% homologous, at least about 90% homologous, at least about 91% homologous, at least about 92% homologous, at least about 93% homologous, at least about 94% homologous, at least about 95% homologous, at least about 96% homologous, at least about 97% homologous, at least about 98% homologous, or at least about 99% homologous to an amino acid sequence disclosed herein.

The invention should also be construed to include any form of a fragment having a substantial length of an amino acid sequence disclosed herein. In one embodiment, a fragment is at least about 50% of the length, at least about 70% of the length, at least about 80% of the length, at least about 90% of the length, at least about 91% of the length, at least about 92% of the length, at least about 93% of the length, at least about 94% of the length, at least about 95% of the length, at least about 96% of the length, at least about 97% of the length, at least about 98% of the length, or at least about 99% of the length of an amino acid sequence disclosed herein.

The invention should also be construed to include any form of a fragment of a protein variant, having both substantial homology to and a substantial length of an amino acid sequence disclosed herein. In one embodiment, a fragment of a protein variant is between 50% and 99% homologous to an amino acid sequence disclosed herein and is between 50% and 99% of the length of an amino acid sequence disclosed herein.

The peptide may alternatively be made by recombinant means or by cleavage from a longer protein or peptide. The peptide may be confirmed by amino acid analysis or sequencing.

The variants of the proteins according to the present invention may be (i) one in which one or more of the amino acid residues are substituted with a conserved or non-conserved amino acid residue (e.g., a conserved amino acid residue) and such substituted amino acid residue may or may not be one encoded by the genetic code, (ii) one in which there are one or more modified amino acid residues, e.g., residues that are modified by the attachment of substituent groups, (iii) one in which the protein comprises an alternative splice variant of the proteins or domains described herein, (iv) fragments of the proteins or domains described herein and/or (v) one in which the protein is fused with another protein or peptide, such as a leader or secretory sequence or a sequence which is employed for purification (for example, His-tag) or for detection (for example, Sv5 epitope tag). The fragments include proteins or peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one polypeptide to a sequence of a second polypeptide. Variants are defined to include peptide sequences different from the original sequence, e.g., different from the original sequence in less than 40% of residues per segment of interest, different from the original sequence in less than 25% of residues per segment of interest, different by less than 10% of residues per segment of interest, or different from the original protein sequence in just a few residues per segment of interest and at the same time sufficiently homologous to the original sequence to preserve the functionality of the original sequence. The present invention includes amino acid sequences that are at least 60%, 65%, 70%, 72%, 74%, 76%, 78%, 80%, 90%, 91%, 92%, 93%, 94%, 95%, 96%, 97%, 98% or 99% similar or identical to the original amino acid sequence. The degree of identity between two polypeptides may be determined using computer algorithms and methods that are widely known for the persons skilled in the art. The identity between two amino acid sequences may be determined by using the BLASTP algorithm (BLAST Manual, Altschul, S., et al., NCBI NLM NIH Bethesda, Md. 20894, Altschul, S., et al., J. Mol. Biol. 215: 403-410 (1990)).

The protein of the invention may or may not be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction. A polypeptide or protein of the invention may be phosphorylated using conventional methods such as the method described in Reedijk et al. (The EMBO Journal 11(4):1365, 1992).

The protein of the invention may include unnatural amino acids formed by post-translational modification or by introducing unnatural amino acids during translation. A variety of approaches are available for introducing unnatural amino acids during polypeptide translation.

A protein of the invention may be conjugated with other molecules, such as polyethylene glycol (PEG). This may be accomplished by inserting cysteine mutations or unnatural amino acids that can be modified with a chemically reactive PEG derivative. In one embodiment, the protein is conjugated to other proteins, to prepare fusion proteins. This may be accomplished, for example, by the synthesis of N-terminal or C-terminal fusion proteins provided that the resulting fusion protein retains the functionality of the protein inhibitor described herein.

Cyclic derivatives of the proteins of the invention are also part of the present invention. Cyclization may allow the protein to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic protein which is more flexible than the cyclic proteins containing peptide bond linkages as described above. A more flexible protein may be prepared by introducing cysteines at the right and left position of the polypeptide and forming a disulfide bridge between the two cysteines. The two cysteines are arranged so as not to deform the beta-sheet and turn. The protein is more flexible as a result of the length of the disulfide linkage and the smaller number of hydrogen bonds in the beta-sheet portion. The relative flexibility of a cyclic protein can be determined by molecular dynamics simulations.

In some embodiments, the peptide inhibitor comprises a targeting domain capable of directing the resulting peptide to a desired cellular component or cell type or tissue. In certain embodiments, the peptide inhibitor comprises additional amino acid sequences or domains. The chimeric or fusion proteins are recombinant in the sense that the various components are from different sources, and as such are not found together in nature (i.e., are heterologous).

In one embodiment, the targeting domain can be a membrane spanning domain, a membrane binding domain, or a sequence directing the peptide inhibitor to associate, for example, with vesicles or with the cell surface. In one embodiment, the targeting domain can target a protein to a particular cell type or tissue. For example, the targeting domain can be a cell surface ligand or an antibody against cell surface antigens of a target tissue. A targeting domain may target a peptide inhibitor of the invention to a cellular component.

A protein of the invention may be synthesized by conventional techniques. For example, the proteins may be synthesized by chemical synthesis using solid phase peptide synthesis. These methods employ either solid or solution phase synthesis methods (see for example, J. M. Stewart, and J. D. Young, Solid Phase Peptide Synthesis, 2nd Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis). By way of example, a polypeptide of the invention may be synthesized using 9-fluorenyl methoxycarbonyl (Fmoc) solid phase chemistry with direct incorporation of phosphothreonine as the N-fluorenylmethoxy-carbonyl-O-benzyl-L-phosphothreonine derivative.

N-terminal or C-terminal fusion proteins comprising a peptide or protein of the invention, conjugated with at least one other molecule, may be prepared by fusing, through recombinant techniques, the N-terminal or C-terminal end of the peptide or protein, and the sequence of a selected protein or selectable marker with a desired biological function. The resultant fusion proteins contain the peptide of the invention fused to the selected protein or marker protein as described herein. Examples of proteins which may be used to prepare fusion proteins include immunoglobulins and regions thereof, glutathione-S-transferase (GST), hemagglutinin (HA), and truncated myc.

A protein of the invention may be developed using a biological expression system. The use of these systems allows the production of large libraries of random sequences and the screening of these libraries for sequences that bind to particular proteins. Libraries may be produced by cloning synthetic DNA that encodes random peptide sequences into appropriate expression vectors (see Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

The protein of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

The present invention further encompasses fusion proteins in which the protein of the invention or fragments thereof, are recombinantly fused or chemically conjugated (including both covalent and non-covalent conjugations) to heterologous proteins (i.e., an unrelated protein or portion thereof, e.g., at least 10, at least 20, at least 30, at least 40, at least 50, at least 60, at least 70, at least 80, at least 90 or at least 100 amino acids of the polypeptide) to generate fusion proteins. The fusion does not necessarily need to be direct but may occur through linker sequences.

In one example, a fusion protein in which a protein of the invention or a fragment thereof can be fused to sequences derived from various types of immunoglobulins. For example, a polypeptide of the invention can be fused to a constant region (e.g., hinge, CH₂, and CH₃ domains) of human IgG or IgM molecule, for example, as described herein, so as to make the fused protein or fragments thereof more soluble and stable in vivo. In another embodiment, such fusion proteins can be administered to a subject so as to inhibit interactions between a ligand and its receptors in vivo. Such inhibition of the interaction will block or suppress signal transduction which triggers certain cellular responses.

In one embodiment, the peptide comprises a domain that enhances stability or half-life of the fusion protein. For example, in one embodiment, the domain comprises at least one region of an immunoglobulin, human serum albumin (HSA), or a peptide or antibody fragment that binds to immunoglobulin, HSA, the erythrocyte cell surface, or the neonatal Fc receptor. In one embodiment, the domain comprises a fragment or variant of at least one region of an immunoglobulin. For example, in one embodiment, the domain comprises an Fc region of an immunoglobulin. Exemplary immunoglobulins include, but is not limited to, IgG1, IgG2, IgG3, IgG4, IgM, IgA, IgE, and IgD.

In one aspect, the fusion protein comprises a polypeptide of the invention which is fused to a heterologous signal sequence at its N-terminus. For example, the signal sequence naturally found in the protein of the invention can be replaced by a signal sequence which is derived from a heterologous origin. Various signal sequences are commercially available. For example, the secretory sequences of melittin and human placental alkaline phosphatase (Stratagene; La Jolla, Calif.) are available as eukaryotic heterologous signal sequences. As examples of prokaryotic heterologous signal sequences, the phoA secretory signal (Sambrook, et al., supra; and Current Protocols in Molecular Biology, 1992, Ausubel, et al., eds., John Wiley & Sons) and the protein A secretory signal (Pharmacia Biotech; Piscataway, N.J.) can be listed. Another example is the gp67 secretory sequence of the baculovirus envelope protein (Current Protocols in Molecular Biology, 1992, Ausubel, et al., eds., John Wiley & Sons).

In another embodiment, a protein of the invention can be fused to tag sequences, e.g., a hexa-histidine peptide, such as the tag provided in a pQE vector (QIAGEN, Inc., 9259 Eton Avenue, Chatsworth, Calif., 91311), among others, many of which are commercially available. As described in Gentz, et al., 1989, Proc. Natl. Acad. Sci. USA 86:821-824, for instance, hexa-histidine provides for convenient purification of the fusion protein. Other examples of peptide tags are the hemagglutinin “HA” tag, which corresponds to an epitope derived from the influenza hemagglutinin protein (Wilson, et al., 1984, Cell 37:767) and the “flag” tag (Knappik, et al., 1994, Biotechniques 17(4):754-761). These tags are especially useful for purification of recombinantly produced proteins of the invention.

### Fusion Molecules

In one embodiment, the composition comprises a fusion or conjugate molecule comprising CXCL12, or fragment or variant thereof, fused or conjugated to a targeting domain. In certain embodiments, the targeting domain directs the fusion molecule to bone or a site of bone formation. For example, in one embodiment, the targeting domain comprises a bisphosphonate or an analog thereof. The bisphosphonate can be any bisphosphonate known in the art to target to bone, and are described, for example in Tabatabaei-Malazy et al. (2017, DARU J. Pharm. Sci. 25:2), and Farrell et al., (2018, Bone Reports 9:47-60), which are incorporated herein by reference.

In one embodiment, the bisphosphonate comprises a structure of formula (1):

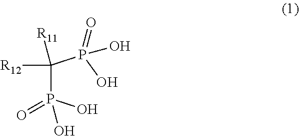

wherein R₁₁ and R₁₂ are each independently selected from the group consisting of hydrogen, halogen, alkyl, aryl, heteroaryl, alkylaryl, alkylheteroaryl, cycloalkyl, heterocyclyl, OR₁₃ and —(CH₂)ₙ—NR₁₃R₁₄;

wherein each occurrence of R₁₃ and R₁₄ is independently selected from the group consisting of hydrogen, alkyl, aryl, heteroaryl, alkylaryl, alkylheteroaryl, cycloalkyl, and heterocycle; and

n is an integer from 0 to 10.

In one embodiment, R₁₁ and R₁₂ are each independently selected from the group consisting of hydrogen, —OH, methyl, Cl, —(CH₂)₂—NH₂, —(CH₂)₅—NH₂, —(CH₂)₃—NH₂, —(CH₂)₂—N(CH₃)((CH₂)₄CH₃),

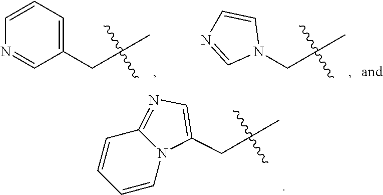 [SMILES: *Cc1cccnc1 ; *Cn1ccnc1 ; *Cc1cnc2ccccn12]

In one embodiment, the bisphosphonate is selected from the group consisting of

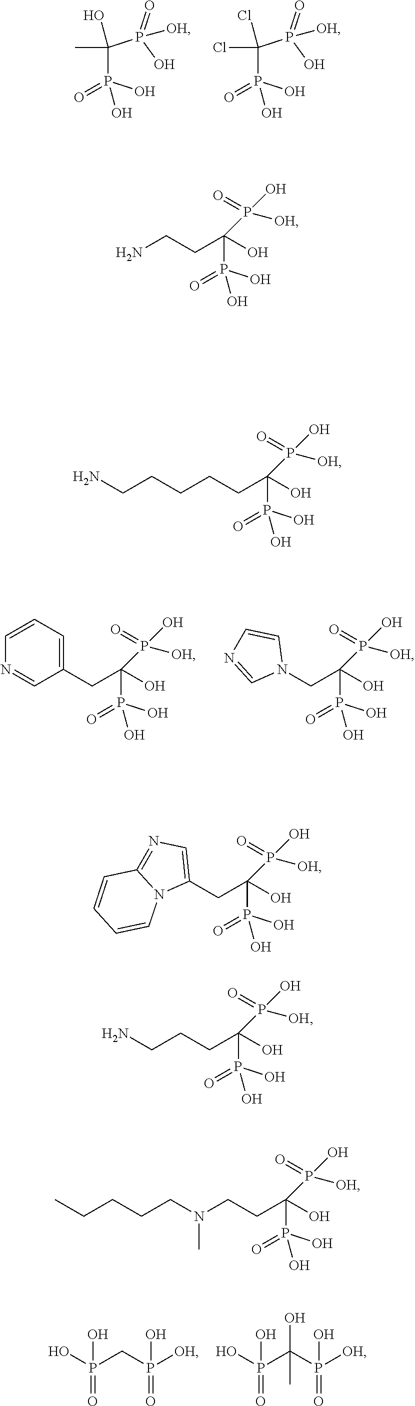

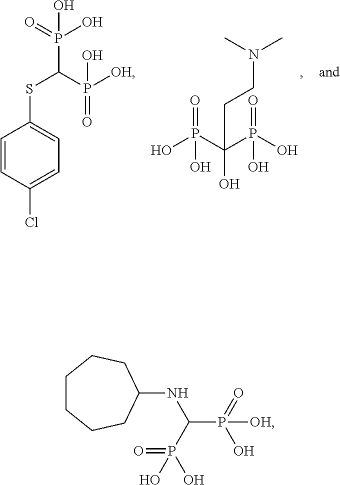 [SMILES: O=P(O)(O)C(Sc1ccc(Cl)cc1)P(=O)(O)O ; CN(C)CCC(O)(P(=O)(O)O)P(=O)(O)O ; O=P(O)(O)C(NC1CCCCCC1)P(=O)(O)O]

or a salt thereof.

In one embodiment, the fusion molecule comprises a CXCL12, or fragment or variant thereof, fused or conjugated to a bisphosphonate, wherein the CXCL12 comprises a non-canonical amino acid and the bisphosphonate is conjugated to the non-canonical amino acid.

In one embodiment, the fusion molecule comprises a CXCL12, or fragment or variant thereof, wherein the CXCL12 comprises a non-canonical amino acid, wherein the non-canonical amino acid comprises a triazole linker, wherein a bisphosphonate is conjugated to the triazole linker.

In one embodiment, the fusion molecule comprises (a) a CXCL12, or fragment or variant thereof, and (2) a bisphosphonate. For example, in one embodiment, the fusion molecule comprises the sequence: KPVSLSYRCPCRFFESHVARANVKHLKILNTPNCALQIVARLKNNNRQVCIDPKL KWIQEYLEKALNKX (SEQ ID NO: 5), wherein X comprises a linker and the bisphosphonate is conjugated to the linker. In one embodiment, X comprises a triazole linker and the bisphosphonate is conjugated to the triazole linker.

In one embodiment, the fusion molecule comprises the sequence: KPVCLCYRCPCRFFESHVARANVKHLKILNTPNCALQIVARLKNNNRQVCIDPK LKWIQEYLEKALNKX (SEQ ID NO: 6), wherein X comprises a linker and the bisphosphonate is conjugated to the linker. In one embodiment, X comprises a triazole linker and the bisphosphonate is conjugated to the triazole linker.

In one embodiment, the fusion molecule comprises the sequence: KPLCVCYRCPCRFFESHVARANVKHLKILNTPNCALQIVARLKNNNRQVCIDPK LKWIQEYLEKALNKX (SEQ ID NO: 7), wherein X comprises a linker and the bisphosphonate is conjugated to the linker. In one embodiment, X comprises a triazole linker and the bisphosphonate is conjugated to the triazole linker.

In one embodiment, the fusion molecule comprises the sequence: MNAKVVVVLVLVLTALCLSDGKPVSLSYRCPCRFFESHVARANVKHLKILNTPN CALQIVARLKNNNRQVCIDPKLKWIQEYLEKALNKX (SEQ ID NO: 8), wherein X comprises a linker and the bisphosphonate is conjugated to the linker. In one embodiment, X comprises a triazole linker and the bisphosphonate is conjugated to the triazole linker.

In one embodiment, the fusion molecule comprises the sequence: MNAKVVVVLVLVLTALCLSDGKPVCLCYRCPCRFFESHVARANVKHLKILNTP NCALQIVARLKNNNRQVCIDPKLKWIQEYLEKALNKX (SEQ ID NO: 9), wherein X comprises a linker and the bisphosphonate is conjugated to the linker. In one embodiment, X comprises a triazole linker and the bisphosphonate is conjugated to the triazole linker.

In one embodiment, the fusion molecule comprises the sequence: MNAKVVVVLVLVLTALCLSDGKPLCVCYRCPCRFFESHVARANVKHLKILNTP NCALQIVARLKNNNRQVCIDPKLKWIQEYLEKALNKX (SEQ ID NO: 10), wherein X comprises a linker and the bisphosphonate is conjugated to the linker. In one embodiment, X comprises a triazole linker and the bisphosphonate is conjugated to the triazole linker.

In one embodiment, the fusion molecule comprises a CXCL12, or fragment or variant thereof, fused or conjugated to a bisphosphonate, wherein the CXCL12 comprises a N-terminal linker and the bisphosphonate is conjugated to the N-terminal linker. In one embodiment, the N-terminal linker comprises a triazole.

In one embodiment, the fusion molecule comprises the sequence: XKPVSLSYRCPCRFFESHVARANVKHLKILNTPNCALQIVARLKNNNRQVCIDPK LKWIQEYLEKALNK (SEQ ID NO: 11), wherein X comprises a linker and the bisphosphonate is conjugated to the linker. In one embodiment, X comprises a triazole linker and the bisphosphonate is conjugated to the triazole linker.

In one embodiment, the fusion molecule comprises the sequence: XKPVCLCYRCPCRFFESHVARANVKHLKILNTPNCALQIVARLKNNNRQVCIDP KLKWIQEYLEKALNK (SEQ ID NO: 12), wherein X comprises a linker and the bisphosphonate is conjugated to the linker. In one embodiment, X comprises a triazole linker and the bisphosphonate is conjugated to the triazole linker.

In one embodiment, the fusion molecule comprises the sequence: XKPLCVCYRCPCRFFESHVARANVKHLKILNTPNCALQIVARLKNNNRQVCIDP KLKWIQEYLEKALNK (SEQ ID NO: 13), wherein X comprises a linker and the bisphosphonate is conjugated to the linker. In one embodiment, X comprises a triazole linker and the bisphosphonate is conjugated to the triazole linker.

In one embodiment, the fusion molecule comprises the sequence: XMNAKVVVVLVLVLTALCLSDGKPVSLSYRCPCRFFESHVARANVKHLKILNTP NCALQIVARLKNNNRQVCIDPKLKWIQEYLEKALNK (SEQ ID NO: 14), wherein X comprises a linker and the bisphosphonate is conjugated to the linker. In one embodiment, X comprises a triazole linker and the bisphosphonate is conjugated to the triazole linker.

In one embodiment, the fusion molecule comprises the sequence: XMNAKVVVVLVLVLTALCLSDGKPVCLCYRCPCRFFESHVARANVKHLKILNT PNCALQIVARLKNNNRQVCIDPKLKWIQEYLEKALNK (SEQ ID NO: 15), wherein X comprises a linker and the bisphosphonate is conjugated to the linker. In one embodiment, X comprises a triazole linker and the bisphosphonate is conjugated to the triazole linker.

In one embodiment, the fusion molecule comprises the sequence: XMNAKVVVVLVLVLTALCLSDGKPLCVCYRCPCRFFESHVARANVKHLKILNT PNCALQIVARLKNNNRQVCIDPKLKWIQEYLEKALNK (SEQ ID NO: 16), wherein X comprises a linker and the bisphosphonate is conjugated to the linker. In one embodiment, X comprises a triazole linker and the bisphosphonate is conjugated to the triazole linker.

As would be understood by one of ordinary skill in the art, the triazole linker includes any organic linker that comprises a triazole group. Non-limiting examples of triazoles include 1,2,3-triazole, 1,2,4-triazole, and 1,3,4-triazole, any of which may optionally be further substituted. In one embodiment, the triazole linker comprises a 1,2,3-triazole or a 1,2,4-triazole. In one embodiment, the triazole linker comprises one or more polyethylene glycol groups. For example, in one embodiment, the linker comprises —(O—CH₂—CH₂)ₙ—, wherein n is an integer from 1 to 10. In one embodiment, the linker comprises a carboxylic acid group.

The CXCL12, or fragment or variant thereof, may be conjugated to a targeting domain using any method known in the art. For example, in one embodiment the fusion molecule is synthesized from a precursor CXCL12 molecule and a precursor bisphosphonate molecule. Accordingly, the present invention also provides precursor CXCL12 molecules and precursor bisphosphonate molecules.

In one embodiment, the precursor CXCL12 molecule comprises a reactive moiety and the precursor bisphosphonate molecule comprises a reactive site which, under mild conditions, permits conjugation of the targeting domain to CXCL12 or to a fragment of CXCL12.

In one embodiment, the precursor CXCL12 molecule comprises a reactive site and the precursor bisphosphonate molecule comprises a reactive moiety which, under mild conditions, permits conjugation of the targeting domain to CXCL12 or to a fragment of CXCL12.

In a non-limiting example, the reactive moiety is an alkyne, and the reactive site is an azide. Under mild conditions, the alkyne and azide undergo a [3+2] cyclization reaction to produce a triazole, thereby conjugating the targeting domain to CXCL12, or fragment of CXCL12, via the triazole moiety. It should be understood that the reactive moiety and the reactive site are interchangeable, permitting an equivalent conjugation reaction wherein the functionality between the reactive moiety and the reactive site on the biomolecule have been switched. In another non-limiting example, the reactive moiety is an alkyl or aryl bromide or a maleimide. These reactive moieties can react with a sulfur group or an amine on CXCL12, or fragment of CXCL12, in order to conjugate the targeting moiety to CXCL12, or fragment of CXCL12. In addition, alkyl or aryl bromides and maleimides form covalent bonds with cysteine residues in proteins under mild conditions. Other non-limiting examples of reactive moieties include carbonyl groups such as aldehydes or ketones. Aldehydes and ketones may undergo reaction with amines on CXCL12, or fragment of CXCL12, thereby conjugating the targeting moiety to the CXCL12, or fragment of CXCL12.

In one embodiment, the precursor CXCL12 molecule and precursor bisphosphonate molecule are capable of azide-alkyne cycloaddition. Under mild conditions, the alkyne and azide undergo a [3+2] cyclization reaction to produce a triazole, thereby conjugating the bisphosphonate to the CXCL12 molecule via the triazole moiety.

In one embodiment, the precursor CXCL12 molecule comprises an azide moiety. For example, in one embodiment, the precursor CXCL12 molecule comprises a non-canonical amino acid comprising an azide group. Exemplary non-canonical amino acid comprising an azide group include, but are not limited to, azidohomoalanine (AHA). In one embodiment, the precursor CXCL12 molecule comprises the sequence:

(SEQ ID NO: 17)
KPVSLSYRCPCRFFESHVARANVKHLKILNTPNCALQIVARLKNNNRQVC
IDPKLKWIQEYLEKALNKM_{AHA}.

In one embodiment, the precursor CXCL12 molecule comprises the sequence:

(SEQ ID NO: 18)
KPVCL [symbol] YRCPCRFFESHVARANVKHLKILNTPNCALQIVARLKNNNRQVC
IDPKLKWIQEYLEKALNKM_{AHA}.

In one embodiment, the precursor CXCL12 molecule comprises the sequence:

(SEQ ID NO: 19)
KP [symbol] YRCPCRFFESHVARANVKHLKILNTPNCALQIVARLKNNNRQVC
IDPKLKWIQEYLEKALNKM_{AHA}.

In one embodiment, the precursor CXCL12 molecule comprises the sequence:

(SEQ ID NO: 20)
MNAKVVVVLVLVLTALCLSDGKPVSLSYRCPCRFFESHVARANVKHLKIL
NTPNCALQIVARLKNNNRQVCIDPKLKWIQEYLEKALNKM_{AHA}.

In one embodiment, the precursor CXCL12 molecule comprises the sequence:

(SEQ ID NO: 21)
MNAKVVVVLVLVLTALCLSDGKPV [symbol] LCYRCPCRFFESHVARANVKHLKIL
NTPNCALQIVARLKNNNRQVCIDPKLKWIQEYLEKALNKM_{AHA}.

In one embodiment, the precursor CXCL12 molecule comprises the sequence:

(SEQ ID NO: 22)
MNAKVVVVLVLVLTALCLSDGKP [symbol] YRCPCRFFESHVARANVKHLKIL
NTPNCALQIVARLKNNNRQVCIDPKLKWIQEYLEKALNKM_{AHA}.

In one embodiment, the precursor bisphosphonate molecule comprises an alkyne moiety. In one embodiment, the precursor bisphosphonate molecule is a molecule represented by formula (2):

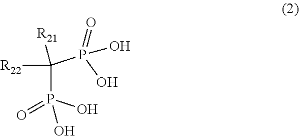

wherein R₂₁ and R₂₂ are each independently selected from the group consisting of hydrogen, halogen, alkyl, aryl, heteroaryl, alkylaryl, alkylheteroaryl cycloalkyl, heterocyclyl, OR₂₃ and —(CH₂)ₘ—NR₂₃R₂₄;

wherein each occurrence of R₂₃ and R₂₄ is independently selected from the group consisting of hydrogen, alkyl, aryl, heteroaryl, alkylaryl, alkylheteroaryl, cycloalkyl,

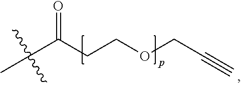

and heterocycle;

m is an integer from 0 to 10; and

p is an integer from 0 to 10,

wherein at least one of R₂₁ and R₂₂ is —(CH₂)ₘ—NR₂₃R₂₄, and at least one of R₂₃ and R₂₄ is

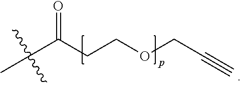

In one embodiment, R₂₁ is —(CH₂)ₘ—NR₂₃R₂₄; R₂₃ is hydrogen, and R₂₄ is

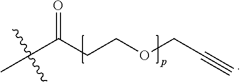

In one embodiment, R₂₁ is —(CH₂)₃—NR₂₃R₂₄; R₂₃ is hydrogen, and R₂₄ is

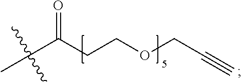

and R₂₂ is hydrogen.

In one embodiment, m is 3.

In one embodiment, p is 5.

In one embodiment, the precursor bisphosphonate molecule is

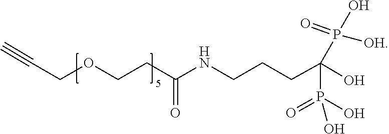

In one embodiment, the precursor CXCL12 molecule comprises an alkyne moiety. For example, in one embodiment, the precursor CXCL12 molecule comprises a non-canonical amino acid comprising an alkyne group. Exemplary non-canonical amino acid comprising an azide group include, but are not limited to, homopropargylglycine. In one embodiment, the CXCL12 N-terminal amine comprises an alkyne group. For example, in one embodiment, the CXCL12 N-terminal amine comprises an ethynylbenzyl.

In one embodiment, the precursor bisphosphonate comprises an azide moiety. In one embodiment, the precursor bisphosphonate molecule is a molecule represented by formula (3):

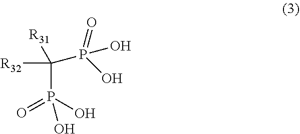

wherein R₃₁ and R₃₂ are each independently selected from the group consisting of hydrogen, halogen, alkyl, aryl, heteroaryl, alkylaryl, alkylheteroaryl, cycloalkyl, heterocyclyl, OR₃₃ and —(CH₂)ₘ—NR₃₃R₃₄;

wherein each occurrence of R₃₃ and R₃₄ is independently selected from the group consisting of hydrogen, alkyl, aryl, heteroaryl, alkylaryl, alkylheteroaryl cycloalkyl,

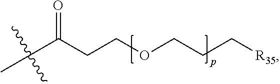

and heterocycle;

m is an integer from 0 to 10;

p is an integer from 0 to 250; and

R₃₅ is selected from the group consisting of N₃ and

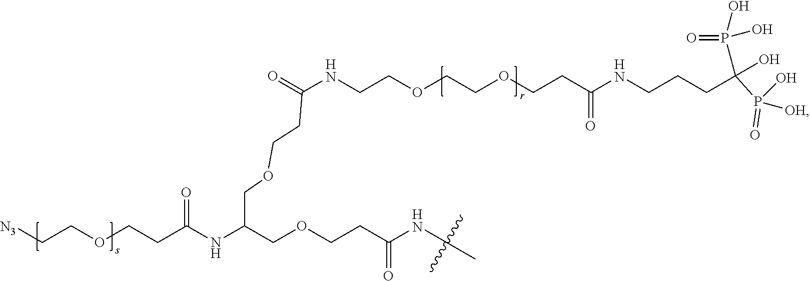

wherein r is an integer from 0 to 250 and s is an integer from 0 to 250.

wherein at least one of R₃₁ and R₃₂ is —(CH₂)ₘ—NR₃₃R₃₄, and at least one of R₃₃ and R₃₄ is

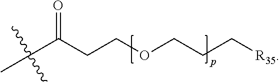

In one embodiment, R₃₁ is —(CH₂)ₘ—N₃₃R₃₄; R₃₂ is hydrogen, and R₃₄ is

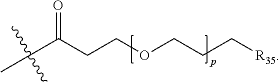

In one embodiment, R₃₁ is —(CH₂)₃—NR₃₃R₃₄; R₃₂ is hydrogen, and R₃₄ is

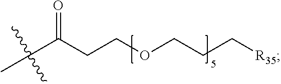

and R₃₂ is hydrogen.

In one embodiment, the precursor bisphosphonate molecule is

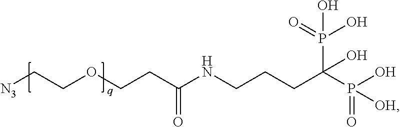

wherein q is an integer from 4 to 250.

In one embodiment, the precursor bisphosphonate molecule is

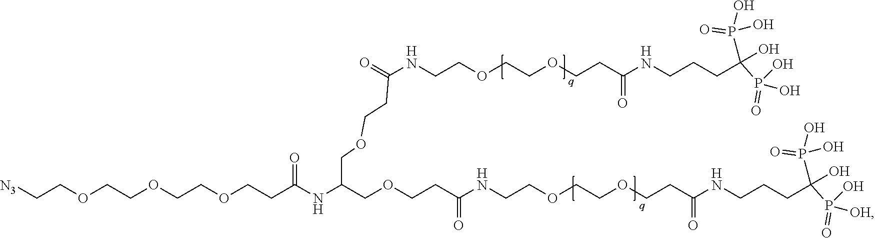

wherein each occurrence of q is independently an integer from 4 to 250.

### Nucleic Acid Molecules

In one embodiment, the present invention provides a composition comprising an isolated nucleic acid sequence encoding a peptide or protein described herein. For example, in one embodiment, the composition comprises an isolated nucleic acid molecule encoding a protein or peptide that activates, increases or enhances CXCL12 expression, activity or both.

In one embodiment, the composition comprises an isolated nucleic acid sequence encoding a biologically functional fragment of a peptide or protein described herein. As would be understood in the art, a biologically functional fragment is a portion or portions of a full-length sequence that retains a biological function of the full-length sequence.

In various embodiments, the isolated nucleic acid sequence encodes a protein or peptide comprising an amino acid sequence selected from the group consisting of SEQ ID NO: 1, SEQ ID NO: 2, SEQ ID NO: 3, and SEQ ID NO: 4

Further, the invention encompasses an isolated nucleic acid encoding a polypeptide having substantial homology to a protein or peptide disclosed herein. In some embodiments, the isolated nucleic acid sequence encodes protein or peptide having at least 75%, 80%, 85%, 90%, 91%, 92% 93%, 94%, 95%, 96%, 97%, 98%, or 99% sequence homology with an amino acid sequence selected from SEQ ID NO: 1, SEQ ID NO: 2, SEQ ID NO: 3, and SEQ ID NO: 4.

The isolated nucleic acid sequence encoding a peptide inhibitor can be obtained using any of the many recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The isolated nucleic acid may comprise any type of nucleic acid, including, but not limited to DNA, cDNA, and RNA. For example, in one embodiment, the composition comprises an isolated DNA molecule, including for example, an isolated cDNA molecule, encoding a protein inhibitor or functional fragment thereof. In one embodiment, the composition comprises an isolated RNA molecule encoding a protein inhibitor or a functional fragment thereof.

The nucleic acid molecules of the present invention can be modified to improve stability in serum or in growth medium for cell cultures. Modifications can be added to enhance stability, functionality, and/or specificity and to minimize immunostimulatory properties of the nucleic acid molecule of the invention. For example, in order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect function of the molecule.

In one embodiment of the present invention the nucleic acid molecule may contain at least one modified nucleotide analogue. For example, the ends may be stabilized by incorporating modified nucleotide analogues.

Non-limiting examples of nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In exemplary backbone-modified ribonucleotides the phosphoester group connecting to adjacent ribonucleotides is replaced by a modified group, e.g., of phosphothioate group. In exemplary sugar-modified ribonucleotides, the 2′ OH-group is replaced by a group selected from H, OR, R, halo, SH, SR, NH₂, NHR, NR2 or ON, wherein R is C1-C6 alkyl, alkenyl or alkynyl and halo is F, Cl, Br or I.

Other examples of modifications are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. The above modifications may be combined.

In some instances, the nucleic acid molecule comprises at least one of the following chemical modifications: 2′-H, 2′-O-methyl, or 2′-OH modification of one or more nucleotides. In some embodiments, a nucleic acid molecule of the invention can have enhanced resistance to nucleases. For increased nuclease resistance, a nucleic acid molecule, can include, for example, 2′-modified ribose units and/or phosphorothioate linkages. For example, the 2′ hydroxyl group (OH) can be modified or replaced with a number of different “oxy” or “deoxy” substituents. For increased nuclease resistance the nucleic acid molecules of the invention can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleobase modifications such as 2-amino-A, 2-thio (e.g., 2-thio-U), G-clamp modifications, can also increase binding affinity to a target. In one embodiment, the nucleic acid molecule includes a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In one embodiment, the nucleic acid molecule includes at least one 2′-O-methyl-modified nucleotide, and in some embodiments, all of the nucleotides of the nucleic acid molecule include a 2′-O-methyl modification.

Nucleic acid agents discussed herein include otherwise unmodified RNA and DNA as well as RNA and DNA that have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, for example as occur naturally in the human body. The art has referred to rare or unusual, but naturally occurring, RNAs as modified RNAs, see, e.g., Limbach et al. (Nucleic Acids Res., 1994, 22:2183-2196). Such rare or unusual RNAs, often termed modified RNAs, are typically the result of a post-transcriptional modification and are within the term unmodified RNA as used herein. Modified RNA, as used herein, refers to a molecule in which one or more of the components of the nucleic acid, namely sugars, bases, and phosphate moieties, are different from that which occur in nature, for example different from that which occurs in the human body. While they are referred to as “modified RNAs” they will of course, because of the modification, include molecules that are not, strictly speaking, RNAs. Nucleoside surrogates are molecules in which the ribophosphate backbone is replaced with a non-ribophosphate construct that allows the bases to be presented in the correct spatial relationship such that hybridization is substantially similar to what is seen with a ribophosphate backbone, e.g., non-charged mimics of the ribophosphate backbone.

Modifications of the nucleic acid of the invention may be present at one or more of, a phosphate group, a sugar group, backbone, N-terminus, C-terminus, or nucleobase.

The present invention also includes a vector in which the isolated nucleic acid of the present invention is inserted. The art is replete with suitable vectors that are useful in the present invention.

Therefore, in another aspect, the invention relates to a vector, comprising the nucleotide sequence of the invention or the construct of the invention. The choice of the vector will depend on the host cell in which it is to be subsequently introduced. In a particular embodiment, the vector of the invention is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with the present invention to produce polynucleotides, or their cognate polypeptides. Many such systems are commercially and widely available.

In brief summary, the expression of natural or synthetic nucleic acids encoding a protein inhibitor is typically achieved by operably linking a nucleic acid encoding the protein inhibitor or portions thereof to a promoter, and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The vectors of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The isolated nucleic acid of the invention can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (2012, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

Further, the expression vector may be provided to a cell in the form of a viral vector. Viral vector technology is well known in the art and is described, for example, in Sambrook et al. (2012), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

By way of illustration, the vector in which the nucleic acid sequence is introduced can be a plasmid, which is or is not integrated in the genome of a host cell when it is introduced in the cell. Illustrative, non-limiting examples of vectors in which the nucleotide sequence of the invention or the gene construct of the invention can be inserted include a tet-on inducible vector for expression in eukaryote cells.

The vector may be obtained by conventional methods known by persons skilled in the art (Sambrook et al., 2012). In a particular embodiment, the vector is a vector useful for transforming animal cells.

In one embodiment, the recombinant expression vectors may also contain nucleic acid molecules, which encode a peptide or protein of invention, described elsewhere herein.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In one embodiment, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method.

In some embodiments, the vector also includes conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The recombinant expression vectors may also contain a selectable marker gene, which facilitates the selection of transformed or transfected host cells. Suitable selectable marker genes are genes encoding proteins such as G418 and hygromycin, which confer resistance to certain drugs, β-galactosidase, chloramphenicol acetyltransferase, firefly luciferase, or an immunoglobulin or portion thereof such as the Fc portion of an immunoglobulin, such as IgG. The selectable markers may be introduced on a separate vector from the nucleic acid of interest.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

Enhancer sequences found on a vector also regulates expression of the gene contained therein. Typically, enhancers are bound with protein factors to enhance the transcription of a gene. Enhancers may be located upstream or downstream of the gene it regulates. Enhancers may also be tissue-specific to enhance transcription in a specific cell or tissue type. In one embodiment, the vector of the present invention comprises one or more enhancers to boost transcription of the gene present within the vector.

In order to assess the expression of a protein inhibitor, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a peptide or protein into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York).

Biological methods for introducing a peptide or protein of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a peptide or protein into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular polypeptide, e.g., by immunological means (ELISAs and Western blots) or by assays described herein to identify agents falling within the scope of the invention.

### Inhibitors of Negative Regulator of CXCL12

In certain embodiments, the modulator comprises an inhibitor of a negative regulator of CXCL12, thereby increasing CXCL12 activity. In some embodiments, the modulator is nucleic acid. In various embodiments, the modulator is an siRNA, miRNA, shRNA, or an antisense molecule, which inhibits a negative regulator of CXCL12, thereby increasing CXCL12 activity. In one embodiment, the nucleic acid comprises a promoter/regulatory sequence such that the nucleic acid is capable of directing expression of the inhibitor nucleic acid. Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In another aspect of the invention, a negative regulator of CXCL12, can be inhibited by way of inactivating and/or sequestering the negative regulator. As such, inhibiting the activity of the negative regulator can be accomplished by using a transdominant negative mutant.

In one embodiment, siRNA is used to decrease the level of a negative regulator of CXCL12. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, P A (2003); and Gregory J. Hannon, Ed., RNAi A Guide to Gene Silencing, Cold Spring Harbor Laboratory Press, Cold Spring Harbor, N.Y. (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216.

In another aspect, the invention includes a vector comprising an siRNA or antisense nucleic acid. In one embodiment, the siRNA or antisense polynucleotide is capable of inhibiting the expression of a target polypeptide, wherein the target polypeptide is a negative regulator of CXCL12. The incorporation of a desired polynucleotide into a vector and the choice of vectors is well-known in the art as described in, for example, Sambrook et al. (2012), and in Ausubel et al. (1997), and elsewhere herein.

In some embodiments, the expression vectors described herein encode a short hairpin RNA (shRNA) inhibitor. shRNA inhibitors are well known in the art and are directed against the mRNA of a target, thereby decreasing the expression of the target. In some embodiments, the encoded shRNA is expressed by a cell, and is then processed into siRNA. For example, in certain instances, the cell possesses native enzymes (e.g., dicer) that cleaves the shRNA to form siRNA.

The siRNA, shRNA, or antisense nucleic acid can be cloned into a number of types of vectors as described elsewhere herein. For expression of the siRNA or antisense polynucleotide, at least one module in each promoter functions to position the start site for RNA synthesis.

In order to assess the expression of the siRNA, shRNA, or antisense nucleic, the expression vector to be introduced into a cell can also contain either a selectable marker gene or a reporter gene or both to facilitate identification and selection of expressing cells from the population of cells sought to be transfected or infected using a viral vector. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl- methyl-, thio- and other modified forms of adenine, cytidine, guanine, thymine, and uridine.

In one embodiment of the invention, an antisense nucleic acid sequence, which is expressed by a plasmid vector is used to inhibit protein expression of a negative regulator of CXCL12. The antisense expressing vector is used to transfect a mammalian cell or the mammal itself, thereby causing reduced endogenous expression of the negative regulator.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between may be about 10 to about 30, nucleotides. In some embodiments, antisense oligomers are about 15 nucleotides. Antisense oligomers about 10 to about 30 nucleotides are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

In one embodiment of the invention, a ribozyme is used to inhibit protein expression of a negative regulator of CXCL12. Ribozymes useful for inhibiting the expression of a target molecule may be designed by incorporating target sequences into the basic ribozyme structure, which are complementary, for example, to the mRNA sequence encoding the negative regulator. Ribozymes targeting the negative regulator, may be synthesized using commercially available reagents (Applied Biosystems, Inc., Foster City, Calif.) or they may be genetically expressed from DNA encoding them.

In one embodiment, the inhibitor of the negative regulator of CXCL12 may comprise one or more components of a CRISPR-Cas system. CRISPR methodologies employ a nuclease, CRISPR-associated (Cas), that complexes with small RNAs as guides (gRNAs) to cleave DNA in a sequence-specific manner upstream of the protospacer adjacent motif (PAM) in any genomic location. CRISPR may use separate guide RNAs known as the crRNA and tracrRNA. These two separate RNAs have been combined into a single RNA to enable site-specific mammalian genome cutting through the design of a short guide RNA. Cas and guide RNA (gRNA) may be synthesized by known methods. Cas/guide-RNA (gRNA) uses a non-specific DNA cleavage protein Cas, and an RNA oligo to hybridize to target and recruit the Cas/gRNA complex. In one embodiment, a guide RNA (gRNA) targeted to a gene encoding the negative regulator, and a CRISPR-associated (Cas) peptide form a complex to induce mutations within the targeted gene. In one embodiment, the inhibitor comprises a gRNA or a nucleic acid molecule encoding a gRNA. In one embodiment, the inhibitor comprises a Cas peptide or a nucleic acid molecule encoding a Cas peptide.

In some embodiments, the inhibitor of a negative regulator of CXCL12 is an antibody, or antibody fragment. In some embodiments, the inhibitor is an antibody, or antibody fragment, that specifically binds to the negative regulator. That is, the antibody can inhibit the negative regulator to provide a beneficial effect.

The antibodies may be intact monoclonal or polyclonal antibodies, and immunologically active fragments (e.g., a Fab or (Fab)₂ fragment), an antibody heavy chain, an antibody light chain, humanized antibodies, a genetically engineered single chain Fv molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, humanized antibodies, fragments and chimeras, may be prepared using methods known to those skilled in the art.

The antibody may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding site, therefore, may include six CDRs, comprising the CDR set from each of a heavy and a light chain V region.

The antibody can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH₁ region, a hinge region, a CH₂ region, and a CH₃ region. The light chain polypeptide of the immunoglobulin can include a VL region and CL region.

The antibody can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

The antibody can be a bispecific antibody. The bispecific antibody can bind or react with two antigens, for example, two of the antigens described below in more detail. The bispecific antibody can be comprised of fragments of two of the antibodies described herein, thereby allowing the bispecific antibody to bind or react with two desired target molecules, which may include the antigen, which is described below in more detail, a ligand, including a ligand for a receptor, a receptor, including a ligand-binding site on the receptor, a ligand-receptor complex, and a marker. Bispecific antibodies can comprise a first antigen-binding site that specifically binds to a first target and a second antigen-binding site that specifically binds to a second target, with particularly advantageous properties such as producibility, stability, binding affinity, biological activity, specific targeting of certain T cells, targeting efficiency and reduced toxicity. In some instances, there are bispecific antibodies, wherein the bispecific antibody binds to the first target with high affinity and to the second target with low affinity. In other instances, there are bispecific antibodies, wherein the bispecific antibody binds to the first target with low affinity and to the second target with high affinity. In other instances, there are bispecific antibodies, wherein the bispecific antibody binds to the first target with a desired affinity and to the second target with a desired affinity.

Antibodies can be prepared using intact polypeptides or fragments containing an immunizing antigen of interest. The polypeptide or oligopeptide used to immunize an animal may be obtained from the translation of RNA or synthesized chemically and can be conjugated to a carrier protein, if desired. Suitable carriers that may be chemically coupled to peptides include bovine serum albumin and thyroglobulin, keyhole limpet hemocyanin. The coupled polypeptide may then be used to immunize the animal (e.g., a mouse, a rat, or a rabbit).

### Delivery Vehicles

In one embodiment, the present invention provides a composition comprising delivery vehicle comprising a modulator of CXCL12, as described herein.

Exemplary delivery vehicles include, but are not limited to, microspheres, microparticles, nanoparticles, polymerosomes, liposomes, and micelles. For example, in some embodiments, the delivery vehicle is loaded with peptide inhibitor, or a nucleic acid molecule encoding a peptide inhibitor. In some embodiments, the delivery vehicle provides for controlled release, delayed release, or continual release of its loaded cargo. In some embodiments, the delivery vehicle comprises a targeting moiety that targets the delivery vehicle to a treatment site.

In one embodiment, the delivery vehicle comprises a bisphosphonate. In one embodiment, a bisphosphonate is conjugated to the surface of the delivery vehicle.

Exemplary bisphosphonates include, but are not limited to,

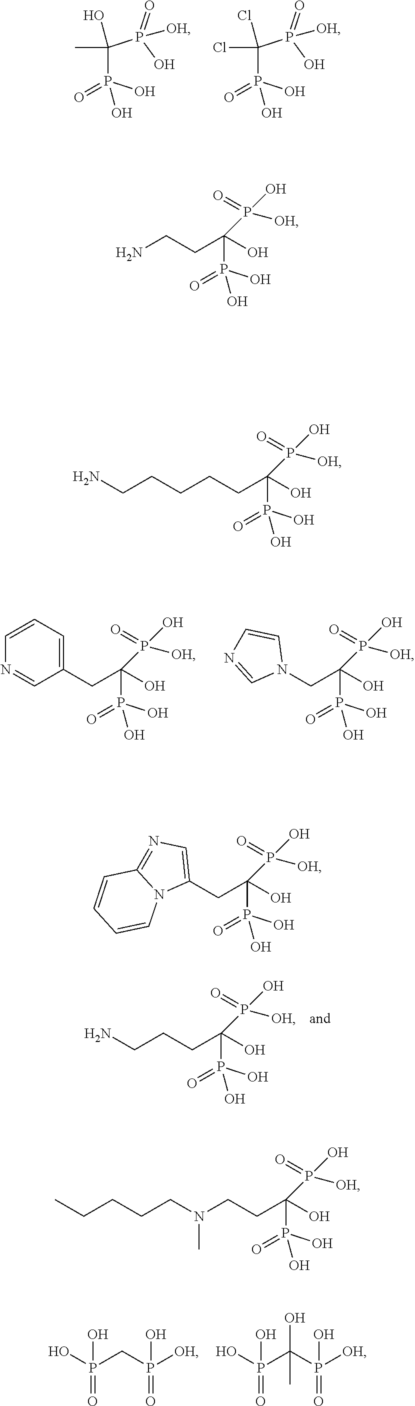

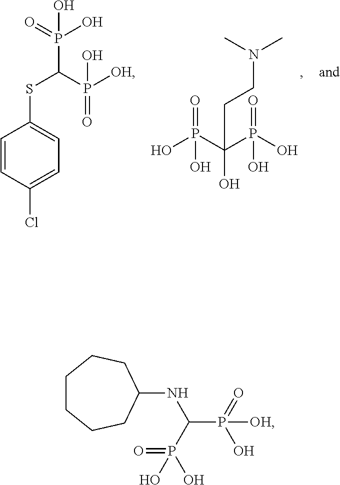 [SMILES: O=P(O)(O)C(Sc1ccc(Cl)cc1)P(=O)(O)O ; CN(C)CCC(O)(P(=O)(O)O)P(=O)(O)O ; O=P(O)(O)C(NC1CCCCCC1)P(=O)(O)O]

and salts thereof.

### Substrates

In one embodiment, the present invention provides a scaffold, substrate, or device comprising a modulator of CXCL12 as described herein.

For example, in some embodiments, the present invention provides a tissue engineering scaffold, including but not limited to, a hydrogel, electrospun scaffold, polymeric matrix, or the like, comprising the modulator. In certain embodiments, the modulator may be coated along the surface of the scaffold, substrate, or device. In certain embodiments, the modulator is encapsulated within the scaffold, substrate, or device.

In certain embodiments, the scaffold comprises a liquid-phase hydrogel comprising the modulator of CXCL12.

### Pharmaceutical Compositions

The present invention also provides pharmaceutical compositions comprising one or more of the compositions described herein. Formulations may be employed in admixtures with conventional excipients, i.e., pharmaceutically acceptable organic or inorganic carrier substances suitable for administration to a treatment site. The pharmaceutical compositions may be sterilized and if desired mixed with auxiliary agents, e.g., lubricants, preservatives, stabilizers, wetting agents, emulsifiers, salts for influencing osmotic pressure buffers, coloring, and/or aromatic substances and the like. They may also be combined where desired with other active agents, e.g., other analgesic agents.

Administration of the compositions of this invention may be carried out, for example, by parenteral, by intravenous, subcutaneous, intramuscular, or intraperitoneal injection, or by infusion or by any other acceptable systemic method.

As used herein, “additional ingredients” include, but are not limited to, one or more of the following: excipients; surface active agents; dispersing agents; inert diluents; granulating and disintegrating agents; binding agents; lubricating agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group: benzyl alcohol, sorbic acid, parabens, imidurea and combinations thereof.

In one embodiment, the composition includes an anti-oxidant and a chelating agent that inhibits the degradation of one or more components of the composition. Exemplary antioxidants for some compounds are BHT, BHA, alpha-tocopherol and ascorbic acid. Exemplary chelating agents include edetate salts (e.g. disodium edetate) and citric acid. The chelating agent is useful for chelating metal ions in the composition that may be detrimental to the shelf life of the formulation. While BHT and disodium edetate may be the antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the compounds or other compositions of the invention in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl para hydroxybenzoates, ascorbic acid, and sorbic acid.

For oral application, particularly suitable are tablets, dragees, liquids, drops, suppositories, or capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, chewing gum, varnishes, sealants, oral and teeth “dissolving strips”, or an emulsion. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin. Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

For oral administration, the compositions of the invention may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400).

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid). Liquid formulations of a pharmaceutical composition of the invention which are suitable for oral administration may be prepared, packaged, and sold either in liquid form or in the form of a dry product intended for reconstitution with water or another suitable vehicle prior to use.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Methods of Modulating CXCL12

In one aspect, the present invention provides a method of activating, increasing, or enhancing CXCL12 expression, activity or both. In one embodiment, the method of modulating CXCL12 comprises administering to a subject or biological system (e.g., a cell, a population of cells, a tissue, an organ, or another system) a composition comprising a modulator of CXCL12, as described elsewhere herein. For example, in one embodiment, the method comprises administering to a subject or biological system a composition comprising a peptide or protein, or a nucleic acid molecule encoding a peptide or protein, wherein the peptide or protein comprises CXCL12, or a fragment or variant thereof. In one embodiment, the method comprises administering to a subject or biological system a composition comprising a peptide or protein, or a nucleic acid molecule encoding a peptide or protein, wherein the peptide or protein comprises at least one of SEQ ID NO: 1, SEQ ID NO: 2, SEQ ID NO: 3, or SEQ ID NO: 4. In one embodiment, the method comprises administering to a subject or biological system a composition comprising a fusion or conjugate molecule, wherein the fusion or conjugate molecule comprises a targeting domain and a modulatory domain. In some embodiments, the modulator domain of the fusion molecule or conjugate molecule comprises CXCL12, or a fragment or variant thereof. In some embodiments, the modulator domain of the fusion molecule or conjugate molecule comprises at least one of SEQ ID NO: 1, SEQ ID NO: 2, SEQ ID NO: 3, SEQ ID NO: 4 SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16. In some embodiments, the fusion molecule or conjugate molecule comprises a targeting domain comprising bisphosphonate or analog thereof.

In some embodiments, the method of modulating CXCL12 is used to promote or enhance osteogenesis or bone formation. In some embodiments, the method of modulating CXCL12 is used to treat or prevent a disease or disorder associated with bone defects or reduced or abnormal bone formation, including but not limited to osteoporosis, idiopathic primary osteoporosis, age-related osteoporosis, glucocorticoid-induced osteoporosis, disuse-related bone loss, Hajdu-Cheney syndrome, osteolysis, post-transplant bone disease, Paget's disease of bone, bone fracture, periodontal disease, and periodontitis.

### Treatment Methods

The present invention provides a method for the treatment or prevention of a disease or disorder associated with bone defects or reduced or abnormal bone formation in a subject in need thereof. The present method may be used to treat or prevent any disease or disorder characterized by bone defects or reduced or abnormal bone formation.

Examples of diseases and disorders that may be treated or prevented by way of the present method include, but are not limited to, osteoporosis, idiopathic primary osteoporosis, age-related osteoporosis, glucocorticoid-induced osteoporosis, disuse-related bone loss, Haj du-Cheney syndrome, osteolysis, post-transplant bone disease, Paget's disease of bone, bone fracture, periodontal disease, and periodontitis.

In some embodiments, compositions of the present invention are co-administered with other therapeutics or prophylactics relevant to the diseases including, but not limited to, teriparatide (Forteo) and abaloparatide (Tymlos) and romosozumab (Evenity), bisphosphonates, bone morphogenetic proteins (BMPs), Wnt proteins, estrogen, selective estrogen receptor modulators (SERMs), parathyroid hormone, parathyroid hormone-related protein (PTHrp) calcitonin, calcium, vitamin D, hormone therapy, hormone-like compounds, RANKL inhibitors, and vascular endothelial growth factor (VEGF).

In some embodiments, compositions of the present invention are administered in a treatment regimen in combination with a load-bearing or weight-bearing exercise regimens. For example, in some embodiments, the compositions enhance load-induced bone formation caused by the load-bearing or weight-bearing exercise regime. Exemplary load bearing or weight-bearing exercise regimes may comprise running, walking, jogging, high-impact aerobic exercises, jumping rope, stair climbing, hiking, tennis, dancing, weight-lifting and functional movements such as lifting one's own body weight.

In some embodiments, the composition of the invention is administered before, during, or after another treatment of the disease or disorder.

In one aspect, the invention provides a method for preventing in a subject, a disease or disorder, by administering to the subject a composition described herein. Subjects at risk for a disease or disorder identified by, for example, any diagnostic or prognostic assay. Administration of a prophylactic agent can occur prior to the manifestation of symptoms characteristic of the disease or disorder, such that the disease or disorder is prevented or delayed in its progression.

Another aspect of the invention pertains to methods of modulating expression or activity of CXCL12 for therapeutic purposes. The modulatory method of the invention involves contacting a cell or subject with a composition described herein that modulates the expression or activity of CXCL12.

In some embodiments, the method comprises administering an effective amount of a composition described herein to a subject diagnosed with, suspected of having, or at risk for developing disease or disorder associated bone defects or reduced or abnormal bone formation. In some aspects, the composition is contacted to a cell or tissue where a condition is present or at risk of developing. In one embodiment, the composition is administered systemically to the subject.

The composition of the invention may be administered to a patient or subject in need in a wide variety of ways. Modes of administration include intraoperatively intravenous, intravascular, intramuscular, subcutaneous, intracerebral, intraperitoneal, soft tissue injection, surgical placement, arthroscopic placement, and percutaneous insertion, e.g., direct injection, cannulation or catheterization. Any administration may be a single application of a composition of invention or multiple applications. Administrations may be to single site or to more than one site in the individual to be treated. Multiple administrations may occur essentially at the same time or separated in time.

Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions of the present invention may be administered in a manner appropriate to the disease to be treated (or prevented). The quantity and frequency of administration will be determined by such factors as the condition of the subject, and the type and severity of the subject's disease, although appropriate dosages may be determined by clinical trials.

When “therapeutic amount” is indicated, the precise amount of the compositions of the present invention to be administered can be determined by a physician with consideration of individual differences in age, weight, disease type, extent of disease, and condition of the patient (subject).

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the compositions of the present invention are administered to a patient by intradermal or subcutaneous injection. In another embodiment, the compositions of the present invention are administered by i.v. injection.

The pharmaceutical compositions useful for practicing the invention may be administered to deliver a dose of from 1 ng/kg/day and 100 mg/kg/day. In one embodiment, the invention envisions administration of a dose which results in a concentration of the compound of the present invention from 1 μM and 10 μM in a mammal.

Typically, dosages which may be administered in a method of the invention to a mammal range in amount from 0.5 μg to about 50 mg per kilogram of body weight of the mammal, while the precise dosage administered will vary depending upon any number of factors, including but not limited to, the type of mammal and type of disease state being treated, the age of the mammal and the route of administration. In one embodiment, the dosage will vary from about 1 μg to about 50 mg per kilogram of body weight of the mammal. In one embodiment, the dosage will vary from about 1 mg to about 10 mg per kilogram of body weight of the mammal.

The compound may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

The administration of a nucleic acid or peptide of the invention to the subject may be accomplished using gene therapy. Gene therapy, which is based on inserting a therapeutic gene into a cell by means of an ex vivo or an in vivo technique. Suitable vectors and methods have been described for genetic therapy in vitro or in vivo, and are known as expert on the matter; see, for example, Giordano, Nature Medicine 2 (1996), 534-539; Schaper, Circ. Res 79 (1996), 911-919; Anderson, Science 256 (1992), 808-813; Isner, Lancet 348 (1996), 370-374; Muhlhauser, Circ. Res 77 (1995), 1077-1086; Wang, Nature Medicine 2 (1996), 714-716; WO94/29469; WO97/00957 or Schaper, Current Opinion in Biotechnology 7 (1996), 635-640 and the references quoted therein. The polynucleotide codifying the polypeptide of the invention can be designed for direct insertion or by insertion through liposomes or viral vectors (for example, adenoviral or retroviral vectors) in the cell. In one embodiment, the cell is a cell of the germinal line, an embryonic cell or egg cell or derived from the same. In some instances, the cell is a core cell. Suitable gene distribution systems that can be used according to the invention may include liposomes, distribution systems mediated by receptor, naked DNA and viral vectors such as the herpes virus, the retrovirus, the adenovirus and adeno-associated viruses, among others. The distribution of nucleic acids to a specific site in the body for genetic therapy can also be achieved by using a biolistic distribution system, such as that described by Williams (Proc. Natl. Acad. Sci. USA, 88 (1991), 2726-2729). The standard methods for transfecting cells with recombining DNA are well known by an expert on the subject of molecular biology, see, for example, WO94/29469; see also supra. Genetic therapy can be carried out by directly administering the recombining DNA molecule or the vector of the invention to a patient or transfecting the cells with the polynucleotide or the vector of the invention ex vivo and administering the transfected cells to the patient.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Recombinant CXCL12 and Mechanical Loading Enhance Cortical Bone Defect Repair

Bone fractures reduce quality of life, increase mortality, and are associated with an economic burden of $19 billion/year in the US [Burge, R., et al., JBMR, 2007. 22(3): p. 465-475]. Improved treatment for bone repair, particularly for non-unions, would provide broad clinical, social and economic benefits. rhBMP-2 is one of the few FDA-approved therapeutic agents used clinically to enhance bone repair. Due to the unpredictable side effect profile of rhBMP-2, new effective and safe pro-osteogenic agents should be investigated. The stem cell homing factor CXCL12 has been shown to regulate fracture healing [Kawakami, Y., et al., JBMR, 2015. 30(1): p. 95-105], though there has not been a direct comparison against BMP-2 treatment. Weight-bearing [Boerckel, J. D., et al., 2011. 108(37): p. E674-E680] and delayed cyclic axial compressive loading [Gardner, M. J., et al., JOR, 2006. 24(8): p. 1679-1686] has also been shown to enhance bone repair, though whether rhCXCL12 and mechanical loading result in a synergistic effect is unknown. To this end, experiments were conducted to measure the therapeutic effect of rhCXCL12 treatment and cyclic compressive loading on monocortical tibial defect repair in mice. Results were compared to rhBMP-2, the clinical gold standard.

Bilateral tibial monocortical defects (1.0 mm, circular) were created in 16-week-old adult C57BL/6 female mice, which were sacrificed at post-surgical day (PSD) 10 (n=3 to 6). This model heals reliably through intramembranous ossification [Carrera, R., et al., JBMR 30 (Suppl 1), 2015]. At the time of surgery, each defect was treated with 5 μg of rhBMP-2 (R&D Systems) or rhCXCL12a (R&D Systems) dissolved in the liquid phase of a hydrogel composed of hyaluronic acid and chitosan (BRTI). Hydrogel alone was used as the vehicle control. Once daily on PSD 5 to 8, the right tibia was subjected to a sinusoidal, compressive axial load (120 cycles at 2 Hz with 6 N peak load) using a mechanical testing system (TA ElectroForce 3200) [Norman, S. C., et al., J Biomech, 2015. 48(1): p. 53-58]. MicroCT analysis: Tibiae were fixed in 4% paraformaldehyde overnight at 4° C., and defects were scanned at a 6-micron voxel size (SkyScan 1172). Newly mineralized bone tissue was quantified in a volume of interest that extended 1 mm from each edge of the defect in the longitudinal direction using an adaptive thresholding routine (CTAn Software, Bruker). Tibiae were decalcified in 9% formic acid and paraffin embedded. Thin sections (7 μm) were stained with modified Movat's pentachrome staining. Student's T-test was used to determine significance for P<0.05.

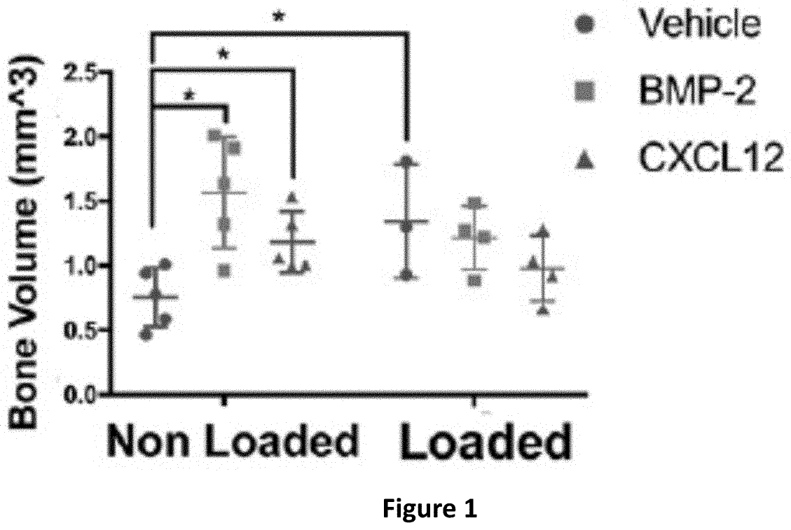

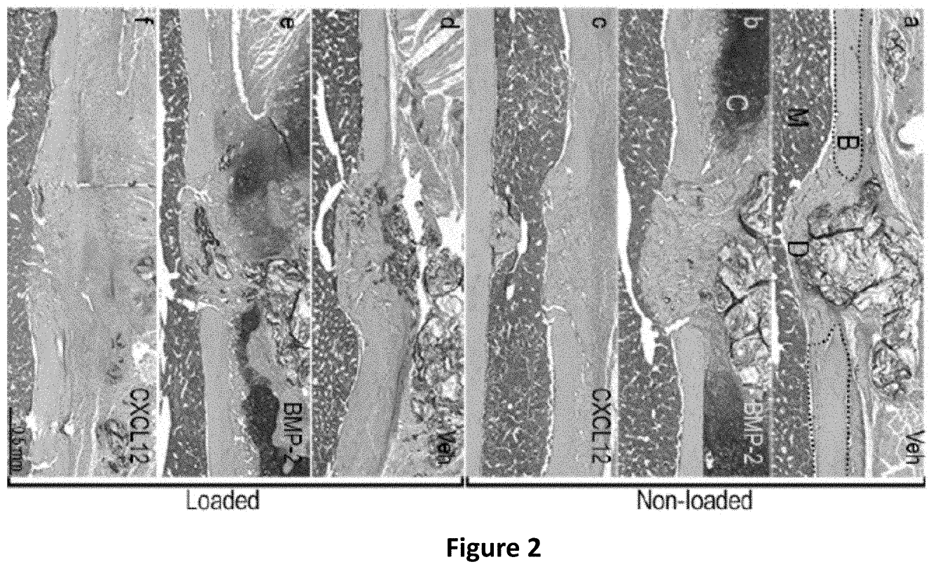

Non-loaded rhCXCL12 and rhBMP-2 treated specimens showed significantly greater new bone volume by microCT compared to vehicle controls (1.57- and 2.08-fold) (FIG. 1). Mechanical loading induced a 1.78-fold increase in new bone volume compared to the non-loaded vehicle controls. All groups subjected to mechanical loading had similar levels of new bone volume, which was comparable to the CXCL12 treatment group. rhBMP-2 (FIG. 2B and FIG. 2E), but not CXCL12 (FIG. 2C and FIG. 2D), promoted cartilage formation.

rhCXCL12α and mechanical loading exhibited comparable therapeutic effects to rhBMP-2 in enhancing bone repair, which may have profound clinical implications. Patients at risk for non-union may be treated with a cost-effective loading exercise regimen, rather than with a cocktail of biologics, which can be associated with unwanted side effects at significant cost to the patient and insurance provider. When exercise alone is insufficient, CXCL12 treatment may be preferred, particularly in cases where excessive cartilage formation is undesirable (e.g., spine fractures). That both mechanical loading and CXCL12 enhance intramembranous bone formation indicates they may activate similar bone anabolic pathways and/or cellular functions. In fact, mechanical loading has been shown to increase CXCL12 expression in osteocytes and bone lining cells [Leucht, P., et al., JOR, 2013. 31(11): p. 1828-1838], suggesting that CXCL12 is a true “mechanobiologic”—a signaling factor whose expression is upregulated in response to physical loading.

### Example 2: Osteocyte-derived CXCL12 is Essential for Load-Induced Bone Formation in Adult Mice

Weight-bearing exercise is an inexpensive means to counteract osteopenia; however, in older patients, mechanical loading alone may be insufficient to enhance bone mass, since load-induced bone formation is attenuated with aging, especially at the periosteal surface. Identifying key cytokines, which positively affect load-induced bone accrual could provide effective therapies to prevent age-associated fracture risk. Previous work showed that the cytokine CXCL12 is necessary for load-induced bone formation in mice ulnae, and that osteocyte-like cells in vitro increase CXCL12 gene expression in response to fluid flow (Leucht et al. JOR. 2013 November; 31(11):1828-38).

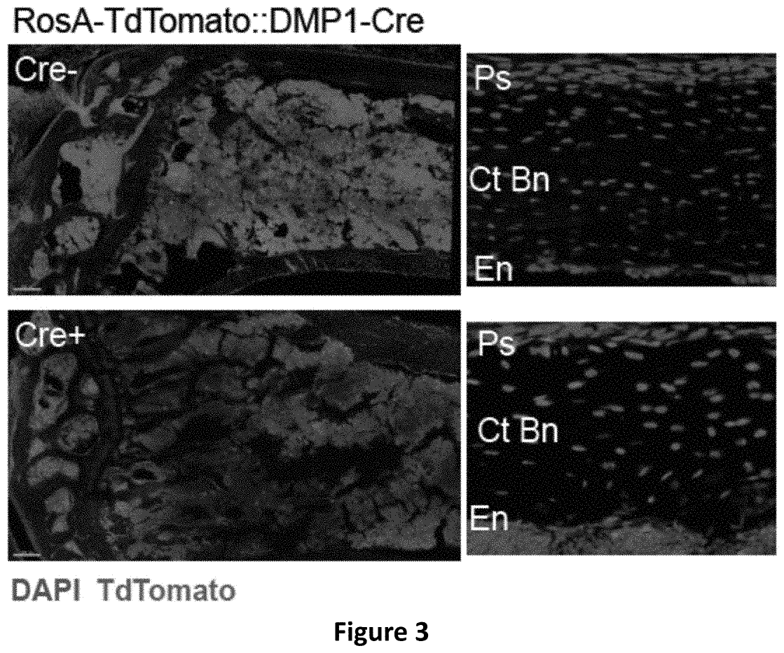

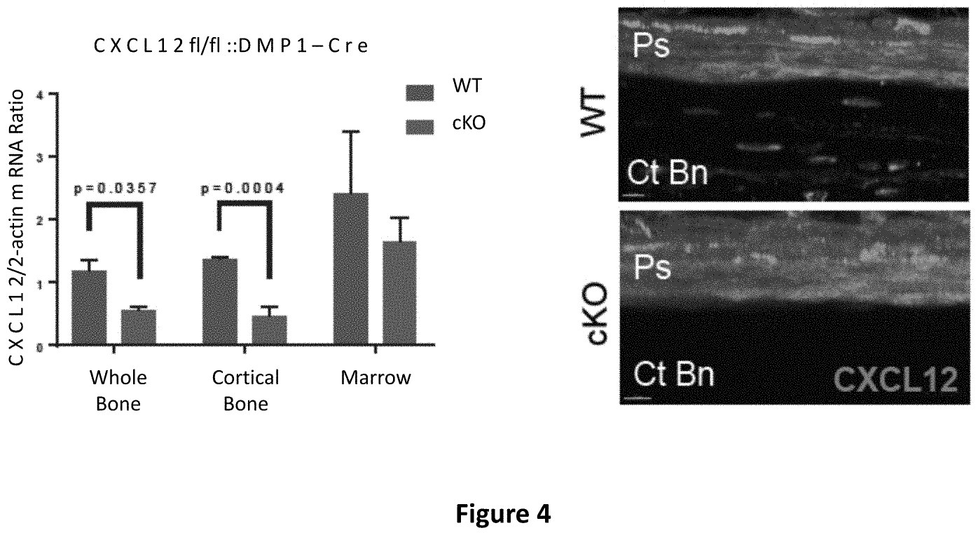

Experiments were conducted to examine whether osteocyte-derived CXCL12 plays a key role in mechanically-driven new bone formation. CXCL12^{fl/fl}::DMP1-Cre mice were generated in a C57Bl/6 background to target deletion in osteocytes. DMP1 expression is shown in FIG. 3, and confirmation of CXCL12 knockout using qRT-PCR and immunofluorescence is shown in FIG. 4.

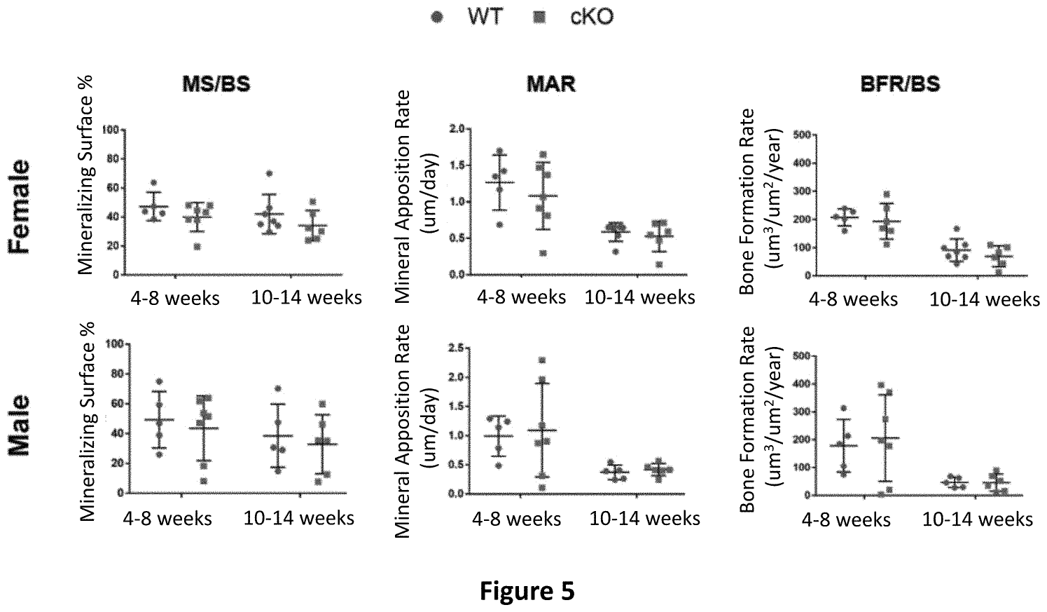

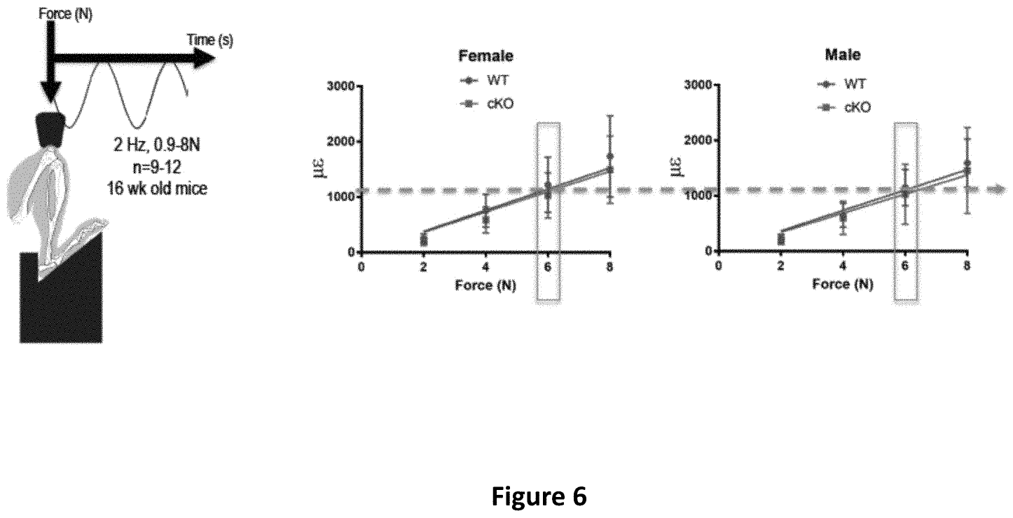

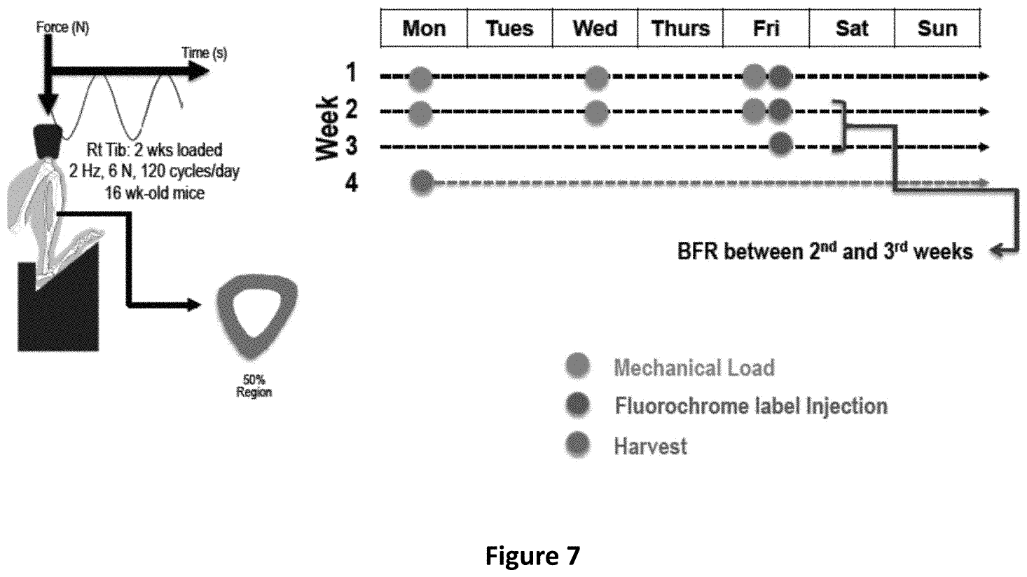

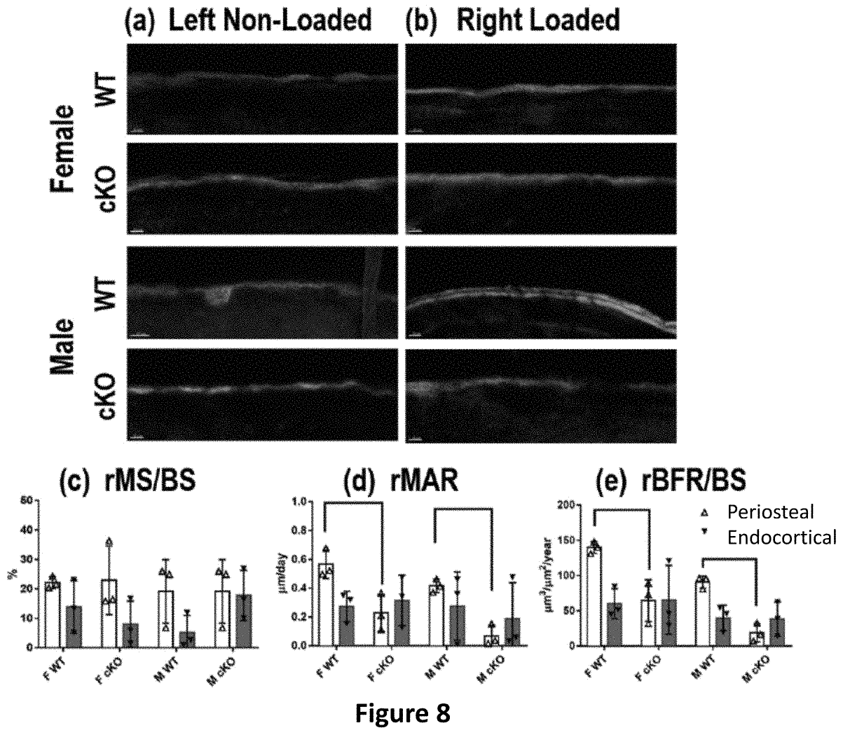

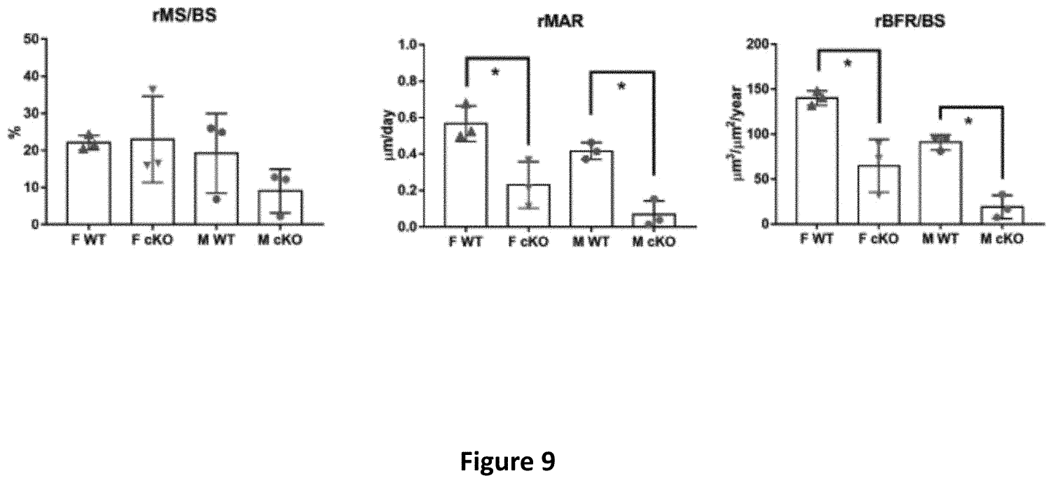

Tibial axial compression (1200με, 2 Hz Haversine) was used for mechanical stimulation. Basal and load-induced periosteal and endosteal bone formation were quantified in male and female mice during development (4-8 wk- and 10-14 wk-old, n=3-8, euthanized 17 wk-old) and starting 16 wk-old (120 cycles, 3 days/wk, 2 wks, n=3-6, euthanized 19 wk-old), respectively. Animals were injected weekly with alternating alizarin red and calcein fluorochrome labels, and their tibiae PMMA embedded and subsequently sectioned at 200 μm. Confocal microscope images of the 50% region of the tibia midshaft, a tibia region previously reported to respond robustly to mechanical loading, were analyzed using ImageJ to measure mineralizing surface (MS/BS,%), mineral apposition rate (MAR, μm/day) and bone formation rate (BFR/BS, μm³/μm²/year). No significant differences were seen in basal bone formation during development (FIG. 5). Further, no difference was seen in strain gage calibration (FIG. 6). A schematic of the experimental plan to investigate load-induced bone formation in WT and CXCL12 knockout mice is shown in FIG. 7. It was observed that relative periosteal load-induced bone formation parameters in the CXCL12fl/fl::DMP1-Cre mice were significantly reduced compared with the wild-type (Cre-mice) values (FIG. 8 and FIG. 9). In particular, rMAR was reduced by 50% in female (p=0.02) and 75% in male (p=0.002) CDC mice. Similarly, rBFR/BS was also reduced by 50% in female (p=0.01) and 70% in male (p=0.001). Conditional deletion of CXCL12 from osteocytes significantly impaired load-induced bone formation. The present studies show that osteocyte derived CXCL12 plays a key role particularly in mechanically driven bone mineral apposition. CXCL12 is a promising candidate to enhance load-induced bone formation.

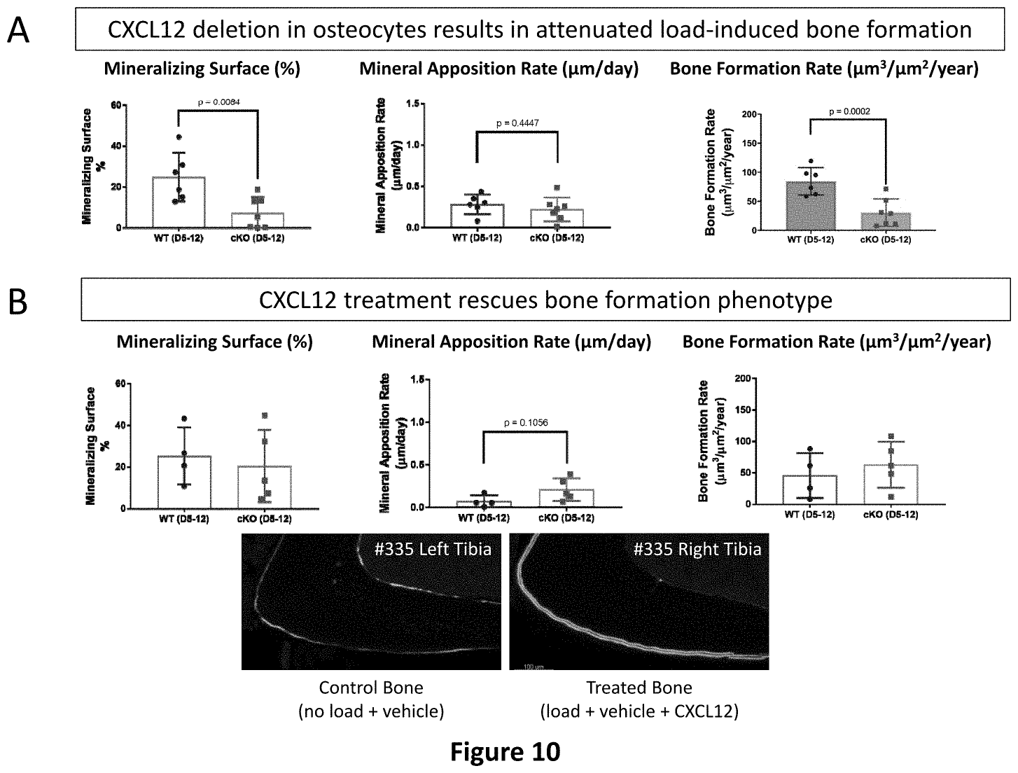

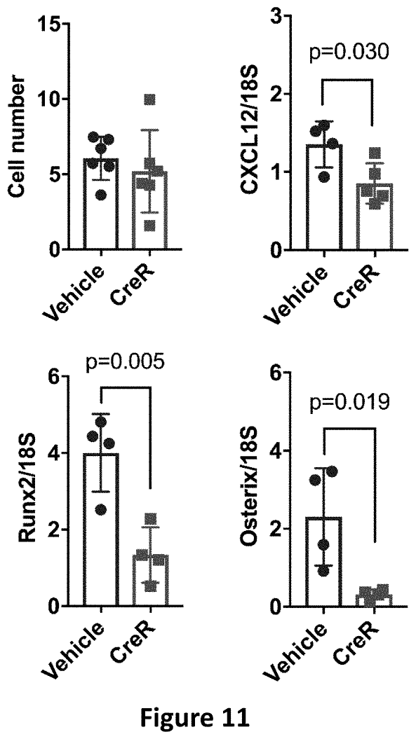

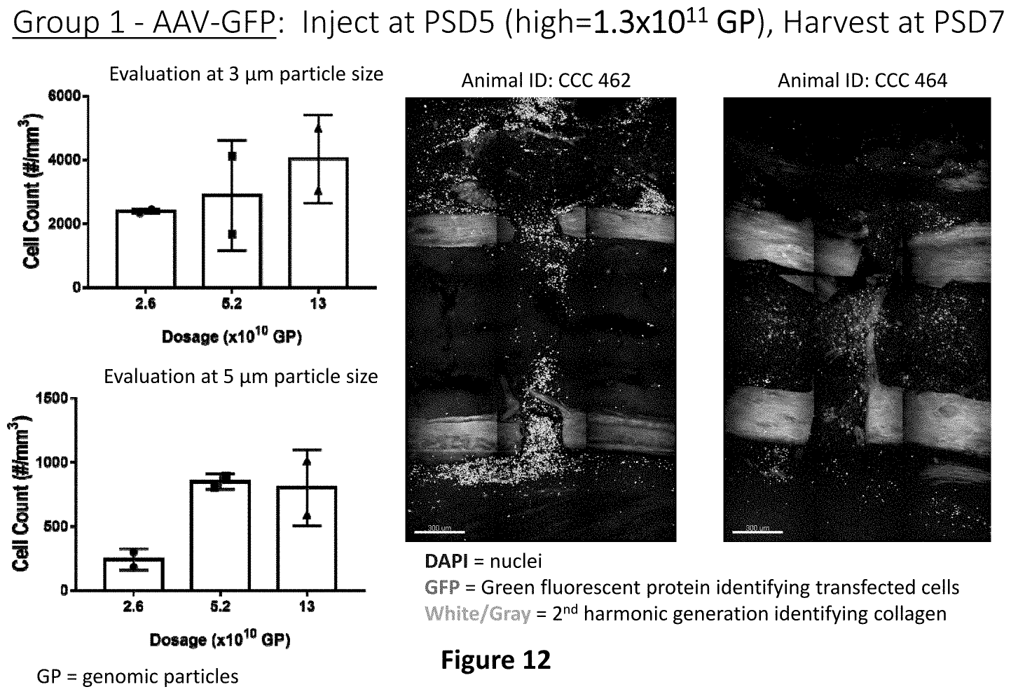

Experiments were then conducted to determine whether CXCL12 treatment can rescue the reduced load-induced bone formation phenotype in CXCL12^{fl/fl}::DMP1-Cre mice. The right tibiae of CXCL12^{fl/fl}::DMP1-Cre mice and littermate controls had their right tibia subjected to axial compressive cyclic loading three times a week for two weeks (days 1, 3, 5, 8, 10 and 12). The left tibia served as an internal control. Recombinant CXCL12+CellMate3D (BRTI Life Sciences #CM-1002) or CellMate3D alone (vehicle control) was injected into the antero-medial aspect of the tibia at mid-length on days 1 and 8. The injection volume was 10 μL with a concentration of 100 μg/mL of CXCL12 (R&D Systems Cat #460-SD-050). Bone fluorochrome labels were injected on days 5 (calcein, 15 mg/kg body weight), 12 (alizarin red, 35 mg/kg body weight) and 19 (calcein, 15 mg/kg body weight). On day 22 the tibiae were harvested and processed for histomorphometric analyses of bone formation rates. It was observed that CXCL12 treatment could rescue the bone phenotype, or more specifically, the ability of bone to respond to mechanical loading as measured by mineralizing surface, mineral apposition rate and bone formation rate (FIG. 10). Experiments were then conducted to determine the mechanism by which CXCL12 exerts influence on the osteogenesis. Primary bone marrow stromal cells, capable of differentiating down the osteogenic, chondrogenic and adipogenic lineages, were isolated from CXCL12 flox/flox mice and cultured in growth media. After expansion, cells were divided into treatment groups and treated with Cre recombinase (CreR), for the purpose of deleting the CXCL12 gene, or vehicle (controls). CXCL12 knockout cells and controls were then cultured in osteogenic media and evaluated for expression of osteogenic genes, including Runx2 and Osterix. CXCL12 knockout cells exhibited significantly lower expression of both Runx2 and Osterix (FIG. 11) suggesting that CXCL12 plays a key role in osteogenic differentiation. Next, a set of experiments was designed to test whether CXCL12 treatment could enhance endochondral bone repair. These experiments were designed to test delivery of AAV-CXCL12 to segmental bone defects. The first experiment, designed to identify a dose of AAV-GFP that resulted in substantial cellular transfection in a healing defect, showed that the mid (5.2×10¹⁰ genomic particles) and high (13×10¹⁰ genomic particles) doses resulted in similar levels of transfection as measured by the number of GFP-positive cells (FIG. 12). Note that the mid and high AAV doses resulted in greater numbers of transfected cells relative to the low dose.

### Example 3: Conjugation of Bisphosphonates to CXCL12

Bisphosphonates can be conjugated via two approaches. In the first approach, CXCL12 is modified to incorporate a non-canonical amino acid having an azide moiety and the bisphosphonate is modified to comprise an alkynyl moiety. The alkyne and azide undergo a [3+2] cyclization reaction to produce a triazole, thereby conjugating the bisphosphonate to the CXCL12 molecule via the triazole moiety. In a second approach, CXCL12 is modified to have an N-terminal alkynyl moiety and the bisphosphonate is modified to comprise an azide moiety. The alkyne and azide undergo a [3+2] cyclization reaction to produce a triazole, thereby conjugating the bisphosphonate to the CXCL12 molecule via the triazole moiety.

First Approach

The non-canonical amino acid azidohomoalanine (AHA) is specifically incorporated at the C-terminus of CXCL12:

(SEQ ID NO: 17)
KPVSLSYRCPCRFFESHVARANVKHLKILNTPNCALQIV ARLKNNNRQV
CIDPKLKWIQ EYLEKALNK [symbol] .

Next, Alkyne-PEG-alendronate is synthesized:

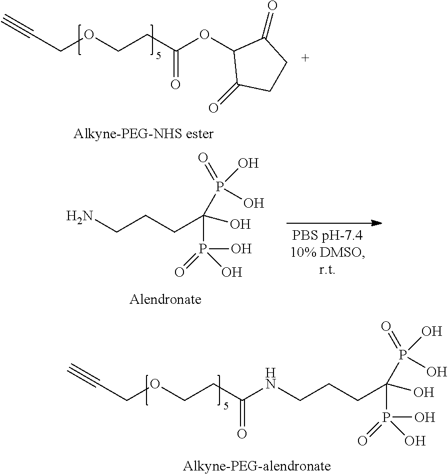

Then, azide-alkyne cycloaddition can be performed to conjugate alkyne-PEG-alendronate to CXCL12.

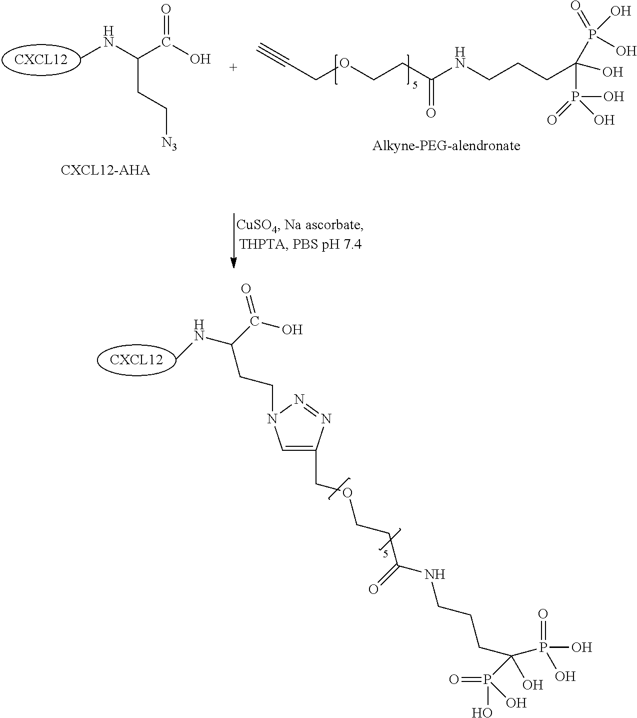

Second Approach

The N-terminus of CXCL12 is reacted with 4-ethynylbenzaldehyde to preserve the N-terminal amine, which would lead to retained bioactivity. Because the reductive alkylation takes place in acidic pH, the lysine group is protonated. So, the reaction would only take place at the N-terminus:

(SEQ ID NO: 1) [symbol]
-KPVSLSYRCPCRFFESHVARANVKHLKILNTPNCALQIVARLKN
NNRQVCIDPKLKWIQEYLEKALNK.

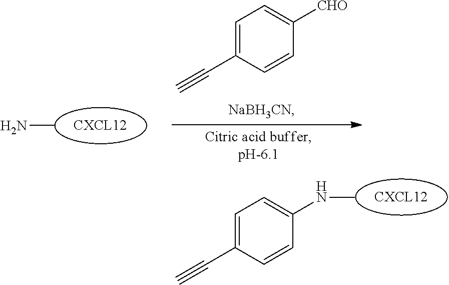

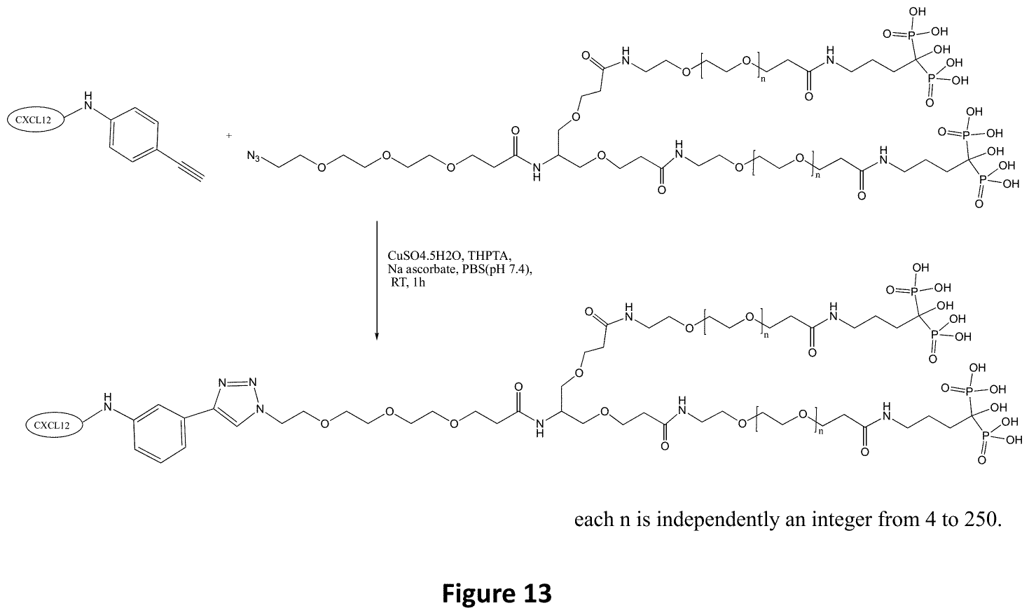

Next, the alkynyl terminated CXCL12 is conjugated with azido-PEG-alendronate by azide-alkyne cycloaddition (FIG. 13).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of treating a disease or disorder associated with bone defects or reduced or abnormal bone formation in a subject in need thereof, wherein the method comprises administering to the subject a composition comprising a modulator of CXCL12.

2. The method of claim 1, wherein the modulator of CXCL12 is at least one of the group consisting of a chemical compound, a protein, a peptide, a peptidomemetic, an antibody, a ribozyme, a small molecule chemical compound, a nucleic acid, a vector, an antisense nucleic acid molecule.

3. The method of claim 2, wherein the modulator of CXCL12 is a protein and the protein comprises at least one sequence of SEQ ID NOs:1-4.

4. The method of claim 1, wherein the modulator of CXCL12 is a fusion molecule, wherein the fusion molecule comprises (a) CXCL12 or a fragment of CXCL12 and (b) a bisphosphonate.

5. The method of claim 3, wherein the fusion molecule comprises a sequence selected from the group consisting of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 15, SEQ ID NO: 15, and SEQ ID NO: 16.

6. The method of claim 4, wherein the bisphosphonate comprises a structure of formula (1): 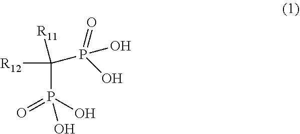
wherein R₁₁ and R₁₂ are each independently selected from the group consisting of hydrogen, halogen, alkyl, aryl, heteroaryl, alkylaryl, alkylheteroaryl, cycloalkyl, heterocyclyl, OR₁₃ and —(CH₂)ₙ—NR₁₃R₁₄;
each occurrence of R₁₃ and R₁₄ is independently selected from the group consisting of hydrogen, alkyl, aryl, heteroaryl, alkylaryl, alkylheteroaryl, cycloalkyl, and heterocycle; and
n is an integer from 0 to 10.

7. The method of claim 6, wherein in formula (1), R₁₁ and R₁₂ are each independently selected from the group consisting of hydrogen, —OH, methyl, Cl, —(CH₂)₂—NH₂, —(CH₂)₃—NH₂, —(CH₂)₅—NH₂, —(CH₂)₂—N(CH₃)((CH₂)₄CH₃),

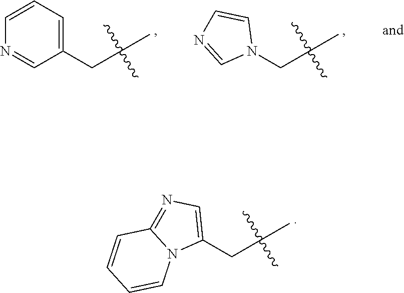 [SMILES: *Cc1cccnc1 ; *Cn1ccnc1 ; *Cc1cnc2ccccn12]

8. The method of claim 6, wherein the bisphosphonate is selected from the group consisting of 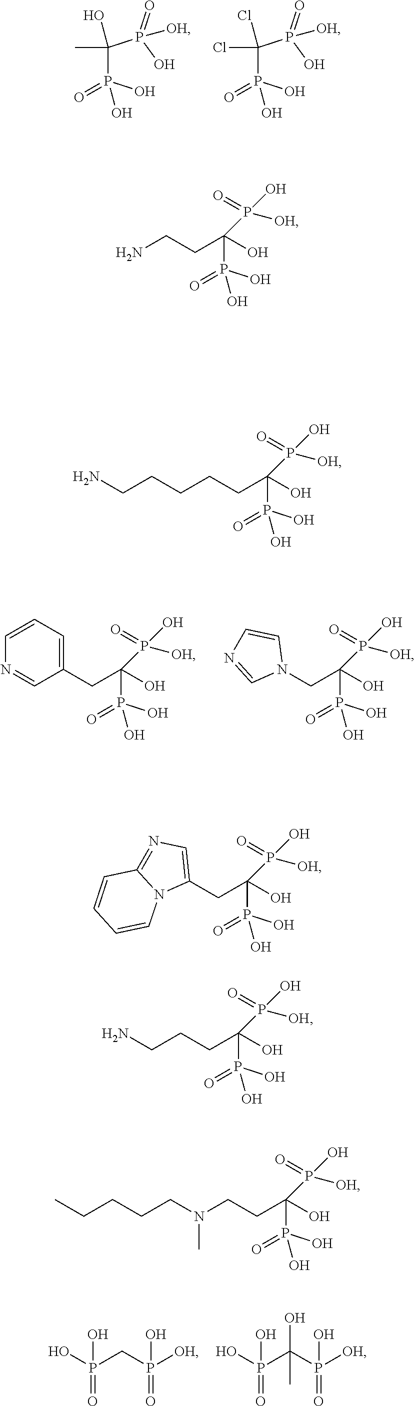 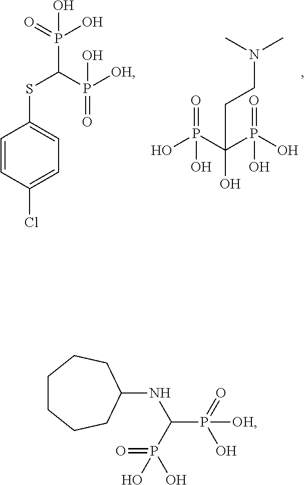 [SMILES: O=P(O)(O)C(Sc1ccc(Cl)cc1)P(=O)(O)O ; CN(C)CCC(O)(P(=O)(O)O)P(=O)(O)O ; O=P(O)(O)C(NC1CCCCCC1)P(=O)(O)O]
and a salt thereof.

9. The method of claim 1, wherein the disease or disorder is selected from the group consisting of osteoporosis, idiopathic primary osteoporosis, age-related osteoporosis, glucocorticoid-induced osteoporosis, Haj du-Cheney syndrome, osteolysis, post-transplant bone disease, Paget's disease of bone, bone fracture, periodontal disease, and periodontitis.

10. A fusion molecule, wherein the fusion molecule comprises (a) CXCL12 or a fragment of CXCL12 and (b) a bisphosphonate.

11. The fusion molecule of claim 10, wherein the fusion molecule comprises a sequence selected from the group consisting of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 15, SEQ ID NO: 15, and SEQ ID NO: 16.

12. The fusion molecule of claim 10, wherein the bisphosphonate comprises a structure of formula (1): 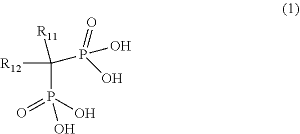
wherein R₁₁ and R₁₂ are each independently selected from the group consisting of hydrogen, halogen, alkyl, aryl, heteroaryl, alkylaryl, alkylheteroaryl, cycloalkyl, heterocyclyl, OR₁₃ and —(CH₂)ₙ—NR₁₃R₁₄;
each occurrence of R₁₃ and R₁₄ is independently selected from the group consisting of hydrogen, alkyl, aryl, heteroaryl, alkylaryl, alkylheteroaryl, cycloalkyl, and heterocycle; and
n is an integer from 0 to 10.

13. The fusion molecule of claim 12, wherein in formula (1), R₁₁ and R₁₂ are each independently selected from the group consisting of hydrogen, —OH, methyl, Cl, —(CH₂)₂—NH₂, —(CH₂)₃—NH₂, —(CH₂)₅—NH₂, —(CH₂)₂—N(CH₃)((CH₂)₄CH₃),

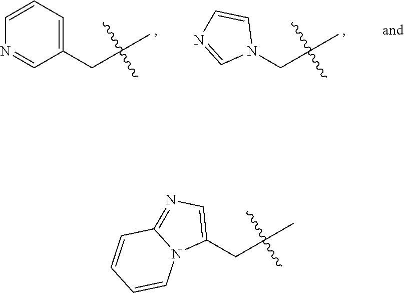 [SMILES: *Cc1cccnc1 ; *Cn1ccnc1 ; *Cc1cnc2ccccn12]

14. The fusion molecule of claim 12, wherein the bisphosphonate is selected from the group consisting of 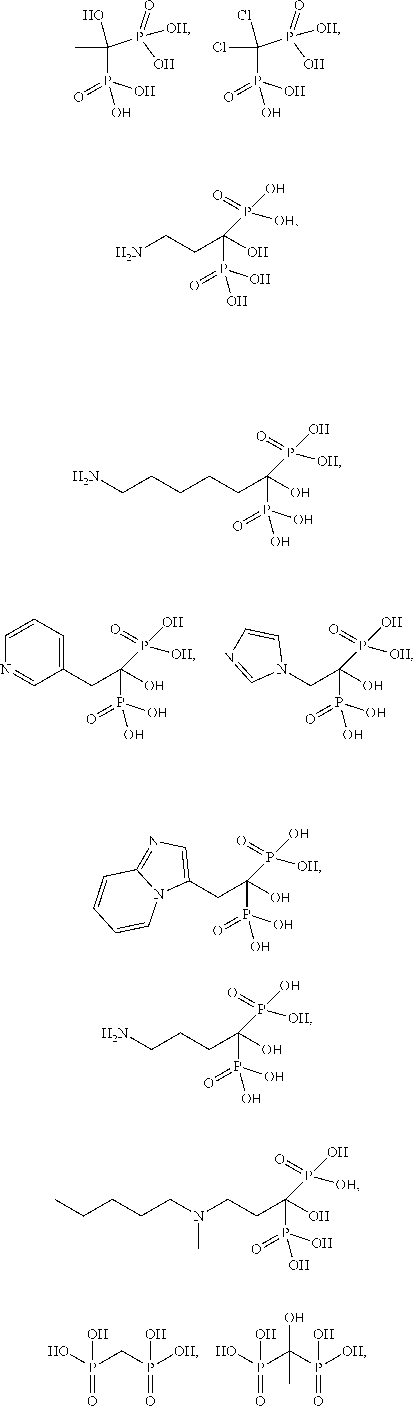 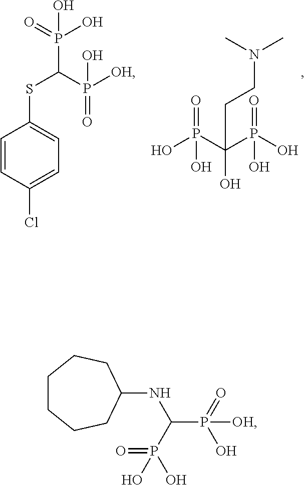 [SMILES: O=P(O)(O)C(Sc1ccc(Cl)cc1)P(=O)(O)O ; CN(C)CCC(O)(P(=O)(O)O)P(=O)(O)O ; O=P(O)(O)C(NC1CCCCCC1)P(=O)(O)O]
and a salt thereof.

15. A method of fusing a CXCL12 or a fragment of CXCL12 to a bisphosphonate, the method comprising:
(a) reacting the N-terminal amine of the CXCL12 or N-terminal amine of the fragment of CXCL12 with 4-ethynylbenzaldehyde to form an alkynyl terminated CXCL12, or incorporating an azidohomoalanine amino acid at the C-terminus of the CXCL12 or the C-terminus of the fragment of CXCL12 to form an alkynyl terminated CXCL12; and
(b) conjugating the alkynyl terminated CXCL12 to an azido-bisphosphonate by azide-alkyne cycloaddition, wherein the conjugation of the alkynyl terminated CXCL12 to the azido-bisphosphonate fuses the CXCL12 or the fragment of CXCL12 to the bisphosphonate.


SEQUENCE LISTING (22 sequences)

SEQ ID NO: 1 - protein, 68 aa, Artificial Sequence
  CXCL12
KPVSLSYRCPCRFFESHVARANVKHLKILNTPNCALQIVARLKNNNRQVCIDPKLKWIQEYLEKALNK

SEQ ID NO: 2 - protein, 68 aa, Artificial Sequence
  CXCL12
KPVCLCYRCPCRFFESHVARANVKHLKILNTPNCALQIVARLKNNNRQVCIDPKLKWIQEYLEKALNK

SEQ ID NO: 3 - protein, 68 aa, Artificial Sequence
  CXCL12
KPLCVCYRCPCRFFESHVARANVKHLKILNTPNCALQIVARLKNNNRQVCIDPKLKWIQEYLEKALNK

SEQ ID NO: 4 - protein, 21 aa, Artificial Sequence
  signal peptide
MNAKVVVVLVLVLTALCLSDG

SEQ ID NO: 5 - protein, 69 aa, Artificial Sequence
  CXCL12 fusion
  misc_feature (69)..(69): Xaa can be any naturally occurring amino acid
KPVSLSYRCPCRFFESHVARANVKHLKILNTPNCALQIVARLKNNNRQVCIDPKLKWIQEYLEKALNKX

SEQ ID NO: 6 - protein, 69 aa, Artificial Sequence
  CXCL12 fusion
  misc_feature (69)..(69): Xaa can be any naturally occurring amino acid
KPVCLCYRCPCRFFESHVARANVKHLKILNTPNCALQIVARLKNNNRQVCIDPKLKWIQEYLEKALNKX

SEQ ID NO: 7 - protein, 69 aa, Artificial Sequence
  CXCL12 fusion
  misc_feature (69)..(69): Xaa can be any naturally occurring amino acid
KPLCVCYRCPCRFFESHVARANVKHLKILNTPNCALQIVARLKNNNRQVCIDPKLKWIQEYLEKALNKX

SEQ ID NO: 8 - protein, 90 aa, Artificial Sequence
  CXCL12 fusion
  misc_feature (90)..(90): Xaa can be any naturally occurring amino acid
MNAKVVVVLVLVLTALCLSDGKPVSLSYRCPCRFFESHVARANVKHLKILNTPNCALQIVARLKNNNRQVCIDPKLKWIQEYLEKALNKX

SEQ ID NO: 9 - protein, 90 aa, Artificial Sequence
  CXCL12 fusion
  misc_feature (90)..(90): Xaa can be any naturally occurring amino acid
MNAKVVVVLVLVLTALCLSDGKPVCLCYRCPCRFFESHVARANVKHLKILNTPNCALQIVARLKNNNRQVCIDPKLKWIQEYLEKALNKX

SEQ ID NO: 10 - protein, 90 aa, Artificial Sequence
  CXCL12 fusion
  misc_feature (90)..(90): Xaa can be any naturally occurring amino acid
MNAKVVVVLVLVLTALCLSDGKPLCVCYRCPCRFFESHVARANVKHLKILNTPNCALQIVARLKNNNRQVCIDPKLKWIQEYLEKALNKX

SEQ ID NO: 11 - protein, 69 aa, Artificial Sequence
  CXCL12 fusion
  misc_feature (1)..(1): Xaa can be any naturally occurring amino acid
XKPVSLSYRCPCRFFESHVARANVKHLKILNTPNCALQIVARLKNNNRQVCIDPKLKWIQEYLEKALNK

SEQ ID NO: 12 - protein, 69 aa, Artificial Sequence
  CXCL12 fusion
  misc_feature (1)..(1): Xaa can be any naturally occurring amino acid
XKPVCLCYRCPCRFFESHVARANVKHLKILNTPNCALQIVARLKNNNRQVCIDPKLKWIQEYLEKALNK

SEQ ID NO: 13 - protein, 69 aa, Artificial Sequence
  CXCL12 fusion
  misc_feature (1)..(1): Xaa can be any naturally occurring amino acid
XKPLCVCYRCPCRFFESHVARANVKHLKILNTPNCALQIVARLKNNNRQVCIDPKLKWIQEYLEKALNK

SEQ ID NO: 14 - protein, 90 aa, Artificial Sequence
  CXCL12 fusion
  misc_feature (1)..(1): Xaa can be any naturally occurring amino acid
XMNAKVVVVLVLVLTALCLSDGKPVSLSYRCPCRFFESHVARANVKHLKILNTPNCALQIVARLKNNNRQVCIDPKLKWIQEYLEKALNK

SEQ ID NO: 15 - protein, 90 aa, Artificial Sequence
  CXCL12 fusion
  misc_feature (1)..(1): Xaa can be any naturally occurring amino acid
XMNAKVVVVLVLVLTALCLSDGKPVCLCYRCPCRFFESHVARANVKHLKILNTPNCALQIVARLKNNNRQVCIDPKLKWIQEYLEKALNK

SEQ ID NO: 16 - protein, 90 aa, Artificial Sequence
  CXCL12 fusion
  misc_feature (1)..(1): Xaa can be any naturally occurring amino acid
XMNAKVVVVLVLVLTALCLSDGKPLCVCYRCPCRFFESHVARANVKHLKILNTPNCALQIVARLKNNNRQVCIDPKLKWIQEYLEKALNK

SEQ ID NO: 17 - protein, 70 aa, Artificial Sequence
  CXCL12 fusion precurosr
  misc_feature (70)..(70): Xaa can be any naturally occurring amino acid
KPVSLSYRCPCRFFESHVARANVKHLKILNTPNCALQIVARLKNNNRQVCIDPKLKWIQEYLEKALNKMX

SEQ ID NO: 18 - protein, 70 aa, Artificial Sequence
  CXCL12 fusion precurosr
  misc_feature (70)..(70): Xaa can be any naturally occurring amino acid
KPVCLCYRCPCRFFESHVARANVKHLKILNTPNCALQIVARLKNNNRQVCIDPKLKWIQEYLEKALNKMX

SEQ ID NO: 19 - protein, 70 aa, Artificial Sequence
  CXCL12 fusion precurosr
  misc_feature (70)..(70): Xaa can be any naturally occurring amino acid
KPLCVCYRCPCRFFESHVARANVKHLKILNTPNCALQIVARLKNNNRQVCIDPKLKWIQEYLEKALNKMX

SEQ ID NO: 20 - protein, 91 aa, Artificial Sequence
  CXCL12 fusion precurosr
  misc_feature (91)..(91): Xaa can be any naturally occurring amino acid
MNAKVVVVLVLVLTALCLSDGKPVSLSYRCPCRFFESHVARANVKHLKILNTPNCALQIVARLKNNNRQVCIDPKLKWIQEYLEKALNKMX

SEQ ID NO: 21 - protein, 91 aa, Artificial Sequence
  CXCL12 fusion precurosr
  misc_feature (91)..(91): Xaa can be any naturally occurring amino acid
MNAKVVVVLVLVLTALCLSDGKPVCLCYRCPCRFFESHVARANVKHLKILNTPNCALQIVARLKNNNRQVCIDPKLKWIQEYLEKALNKMX

SEQ ID NO: 22 - protein, 90 aa, Artificial Sequence
  CXCL12 fusion precurosr
MNAKVVVVLVLVLTALCLSDGKPLCVCYRCPCRFFESHVARANVKHLKILNTPNCALQIVARLKNNNRQVCIDPKLKWIQEYLEKALNKM